SEQUENCE LISTING (41 sequences)

SEQ ID NO: 1 - DNA, 1446 bp, Arabidopsis thaliana
  CDS (1)..(1443)
atggctctcaaggcattagactacgatactctgaatgaaaacgtcaagaagtgtcagtatgccgtaagaggtgaactttatctccgagcttctgagctgcagaaagaaggcaaaaaggttattttcacaaacgttgggaaccctcatgctttaggacagaagccattgacatttcctcgccaggtggttgcgctttgccaagctccgtttctactagatgacccaaatgttggaatgctatttccagctgatgctattgcaagagctaaacattatctttccttgacttcaggcggtttaggtgcttacagtgattcaagaggccttccaggagttaggaaagaggttgctgagttcattcaacggcgtgatgggtatccaagtgacccagaactcatctttctcactgatggagctagcaaaggtgtgatgcaaatcttgaattgtgttatacgcggtaatggagatgggattctagttccggttccacagtatccactttactcagctaccatatcactgttaggtggtactcttgttccttactatcttgatgagtctgaaaactggggacttgatgttgctaaccttcgacaatccgttgctcaggctcgttctcaagggataacagtaagggcaatggtgatcattaaccctgggaacccaactggccagtgtctaagcgaagctaacataagagagatattgaagttctgttataacgagaaactggttcttctgggagacgaggtttatcagcagaacatataccaggatgagcgtccctttatcagctccaagaaggttttgatggaaatgggttcgccgttcagcaaggaagttcagcttgtatcttttcacacagtctctaaaggatattggggtgaatgtggacagcgaggtggatactttgagatgaccaacctccctccaagggttgttgaggagatatacaaggttgcatcaattgccctcagccctaatgtctctgcgcaaatctttatgggtttgatggttaatcctccaaagcctggagacatttcatatgaccagttcgcccgtgaaagcaaggggattcttgaatctttgagaagaagagcaaggctcatgacagatggattcaacagctgcaaaaacgtcgtgtgcaatttcacagaaggtgcaatgtattcgtttcctcaaatacggttaccaacgggagctctccaagctgcaaaacaagctggaaaagtgccagacgttttctactgtctcaagctcttagaagccacaggaatctccacagtacctggctctggatttggacagaaagaaggtgtgttccatctgaggacaacaatcctgccagcagaagatgagatgccggagatcatggatagcttcaagaagttcaacgacgagttcatgactcagtatgataataactttggttattcgaaaatgtga

SEQ ID NO: 2 - protein, 481 aa, Arabidopsis thaliana
MALKALDYDTLNENVKKCQYAVRGELYLRASELQKEGKKVIFTNVGNPHALGQKPLTFPRQVVALCQAPFLLDDPNVGMLFPADAIARAKHYLSLTSGGLGAYSDSRGLPGVRKEVAEFIQRRDGYPSDPELIFLTDGASKGVMQILNCVIRGNGDGILVPVPQYPLYSATISLLGGTLVPYYLDESENWGLDVANLRQSVAQARSQGITVRAMVIINPGNPTGQCLSEANIREILKFCYNEKLVLLGDEVYQQNIYQDERPFISSKKVLMEMGSPFSKEVQLVSFHTVSKGYWGECGQRGGYFEMTNLPPRVVEEIYKVASIALSPNVSAQIFMGLMVNPPKPGDISYDQFARESKGILESLRRRARLMTDGFNSCKNVVCNFTEGAMYSFPQIRLPTGALQAAKQAGKVPDVFYCLKLLEATGISTVPGSGFGQKEGVFHLRTTILPAEDEMPEIMDSFKKFNDEFMTQYDNNFGYSKM

SEQ ID NO: 3 - DNA, 1446 bp, Arabidopsis thaliana
  CDS (1)..(1443)
atgtctctcaaggcgttagactacgagtccttgaatgaaaacgtgaagaattgtcagtatgcagtcagaggtgaactttatcttcgtgcttctgagcttcagaaagaaggcaaaaagattattttcacaaatgttggaaaccctcatgctttaggacagaaacctctgacttttcctcgtcaggtggtttctttatgccaagcaccatttctgttagatgatccaaatgttggtatgatattcccagcagatgctattgcaagagctaagcattatctttccttgacttctggtggtcttggtgcttacagtgactcaagaggtcttccgggagttcggaaagaagtcgctgagttcattgaacggcgtgatggatatccaagcgatccagaactcatatttctaactgatggagcgagcaaaggtgtgatgcaaatcttgaattgtgtcatacgcggtcagaaagacggaattctggttccagttccacagtatccactctactcggctactatatctctgttaggtggtactcttgttccttactatcttgaagagtctgaaaactggggacttgatgttaacaaccttcgccaatctgttgctcaagctcgctctcaaggaataacagtaagggcaatggtgattattaaccccggaaacccaactggccagtgtcttagcgaagctaacataagagagatactacggttctgttgtgatgagagattagttcttctcggagacgaagtgtatcagcaaaatatataccaagatgaacgtccctttatcagttccaagaaggttttgatggatatgggagcaccgatcagcaaggaagttcagctcatatctttccacaccgtttccaaaggatactggggcgaatgtgggcaacggggaggttactttgagatgacaaatatccctcccaggaccgttgaggagatatacaaggtggcctctatagctctcagccccaacgtctctgcgcagatatttatgggtttaatggttagcccaccaaagcctggagacatttcatatgaccaattcgttcgtgagagcaagggaatactagaatcactgagaagaagagcaaggatgatgactgatggattcaacagctgcaaaaacgtcgtctgtaatttcacagaaggtgctatgtattcattccctcaaataaagttgccgtcgaaagcaatccaagcagcaaaacaagccggaaaagtccctgacgttttctactgccttaagctcttagaagccacaggaatctccacagttccaggctctggatttggacaaaaagaaggggtgtttcatttaaggacaacaattctgccagcagaagaagaaatgccagagattatggacagtttcaaaaagttcaatgatgagtttatgtctcagtacgctgataactttggttactccagaatgtga

SEQ ID NO: 4 - protein, 481 aa, Arabidopsis thaliana
MSLKALDYESLNENVKNCQYAVRGELYLRASELQKEGKKIIFTNVGNPHALGQKPLTFPRQVVSLCQAPFLLDDPNVGMIFPADAIARAKHYLSLTSGGLGAYSDSRGLPGVRKEVAEFIERRDGYPSDPELIFLTDGASKGVMQILNCVIRGQKDGILVPVPQYPLYSATISLLGGTLVPYYLEESENWGLDVNNLRQSVAQARSQGITVRAMVIINPGNPTGQCLSEANIREILRFCCDERLVLLGDEVYQQNIYQDERPFISSKKVLMDMGAPISKEVQLISFHTVSKGYWGECGQRGGYFEMTNIPPRTVEEIYKVASIALSPNVSAQIFMGLMVSPPKPGDISYDQFVRESKGILESLRRRARMMTDGFNSCKNVVCNFTEGAMYSFPQIKLPSKAIQAAKQAGKVPDVFYCLKLLEATGISTVPGSGFGQKEGVFHLRTTILPAEEEMPEIMDSFKKFNDEFMSQYADNFGYSRM

SEQ ID NO: 5 - DNA, 1623 bp, Arabidopsis thaliana
  CDS (1)..(1620)
atgcggagattcttgattaaccaagctaaaggtctcgtcgaccattctcgtcgtcaacatcaccacaaaagtccaagctttctctctcctcaacctcgtccccttgcttcttctcctcctgctctgtctcgtttcttctcttctacttcggagatgtctgcttctgattccacttcttctcttcccgttactcttgactccatcaatcccaaggttctgaaatgtgagtatgctgttcgaggagaaattgtcaacattgctcagaagttacaagaagacttgaagactaataaggatgcttatccctttgatgagataatctattgcaacattgggaatcctcaatctcttggtcagctgcctataaagttcttccgtgaggttctcgcattgtgtgaccacgcaagtcttttggatgagtctgaaacccatggtttgttcagtaccgattcaattgaccgagcatggaggattttggaccatattcccggaagagcaactggggcttacagtcatagccagggtatcaagggtttacgtgatgtaattgcagctggaatcgaagcacgtgatggtttccctgctgatccaaatgatattttcttgactgatggtgcaagtccagcggttcacatgatgatgcaacttctcttgagctcagagaaagatggtattctttccccgattcctcagtatccattgtactcggcttcaattgcccttcatggtggatctttggttccgtactatcttgatgaagcaacaggatggggactggaaatatctgaccttaagaagcaactagaggaagctaggtccaagggcatctctgtaagggccttggttgtcataaaccccggtaacccaactgggcaggttcttgcggaagaaaaccagcgtgacattgttaatttctgcaagcaagagggtttggttcttttagctgatgaagtctaccaagaaaatgtttacgtccctgacaaaaagttccattctttcaagaaagtggctcggtctttgggctatggagaaaaagatatctcgttagtctcgttccaatcagtctccaaaggatattatggagaatgtggaaaaagaggaggttacatggaggttactggattcacttctgatgtaagagaacagatatacaaaatggcttctgtgaatctttgttctaacatctctggtcaaattcttgctagccttgtcatgagcccacccaagcctggtgatgactcatatgactcatacatggcagaaagagatggaattctctcatccatggctaaacgtgcaaagactttggaagacgctctcaacagtttagaaggtgttacatgtaacagagccgaaggagcaatgtatctcttcccgcgaattaaccttcctcaaaaggctatcgaagctgctgaggctgaaaaaactgcaccagatgcgttctactgcaaacgccttctcaatgctactggtgtagttgtagtccctggttctggctttggacaggttcctggaacatggcactttagatgcacaatacttccacaagaagacaagattccagcgatagtgaatcgtctgacagagttccacaagagcttcatggacgagttccgcaactaa

SEQ ID NO: 6 - protein, 540 aa, Arabidopsis thaliana
MRRFLINQAKGLVDHSRRQHHHKSPSFLSPQPRPLASSPPALSRFFSSTSEMSASDSTSSLPVTLDSINPKVLKCEYAVRGEIVNIAQKLQEDLKTNKDAYPFDEIIYCNIGNPQSLGQLPIKFFREVLALCDHASLLDESETHGLFSTDSIDRAWRILDHIPGRATGAYSHSQGIKGLRDVIAAGIEARDGFPADPNDIFLTDGASPAVHMMMQLLLSSEKDGILSPIPQYPLYSASIALHGGSLVPYYLDEATGWGLEISDLKKQLEEARSKGISVRALVVINPGNPTGQVLAEENQRDIVNFCKQEGLVLLADEVYQENVYVPDKKFHSFKKVARSLGYGEKDISLVSFQSVSKGYYGECGKRGGYMEVTGFTSDVREQIYKMASVNLCSNISGQILASLVMSPPKPGDDSYDSYMAERDGILSSMAKRAKTLEDALNSLEGVTCNRAEGAMYLFPRINLPQKAIEAAEAEKTAPDAFYCKRLLNATGVVVVPGSGFGQVPGTWHFRCTILPQEDKIPAIVNRLTEFHKSFMDEFRN

SEQ ID NO: 7 - DNA, 1638 bp, Arabidopsis thaliana
  CDS (1)..(1635)
atgcggagattcgtgattggccaagctaaaaatctcatagatcagagtcgtcgtcgtcaacttcatcatcacaaaaatctcagctttgtctctcttattcctcctttttctgctccttccgattcttcatcgcgccacttgtcttcttcttcttcttccgatatgtctgcttctgattcctcttcctctcttcccgttactcttgacaccatcaaccccaaggttatcaaatgtgagtatgctgtccgtggagaaattgtcaacattgctcagaaattgcaagaagatttgaagactaacaaggacgcttatccctttgatgagattatctactgtaatatcgggaatcctcaatctcttggtcaacagcctataacattcttcagagaggttcttgctttatgttcctacacagccctgttggatgagagtgcaacacacggtttgttcaggttcagttctgattcgattgagcgtgcttggaagattctggaccaaattcccgggagagcgactggtgcttacagccacagccagggtatcaaggggttacgtgatgcaattgctgatggaatcgaagcccgtgatggtttccctgctgatcctaatgatatattcatgacagatggtgcaagtccaggggtacatatgatgatgcaacttctcataacttcagagaaagatggaatcctttgtcctattcctcagtatccattgtactcagcttcaattgcccttcacggtggaactttggttccatactaccttgatgaagcatcaggatggggtcttgaaatatctgagctgaagaaacaacttgaagatgctaggtcaaagggcatcactgtgagagctttggctgtcattaaccctggaaacccgacagggcaggttctttcggaagaaaaccagcgtgacgttgttaagttctgcaagcaagagggtttagttcttttagcagacgaggtttatcaagagaatgtctatgtccctgacaaaaagttccattccttcaagaaagtagcccgctctatgggctacggtgagaaggatcttgccttagtctctttccaatctgtctccaaagggtactatggagagtgtgggaaaagaggtggttacatggaggttactggattcacttctgatgtaagagagcagatatacaaaatggcttctgtgaatctttgttccaacatctctggtcaaattcttgctagcctcatcatgagcccacccaagcctggtgacgactcctatgaatcatacatagcagagaaggatggaattctctcatctttggcaagacgtgcaaagactcttgaagaggctctgaacaagctagagggagttacatgcaatagagcagaaggagctatgtatctattcccttgccttcaccttccacaaaaggcaattgcagctgctgaggcggaaaagacagcaccagacaatttctactgcaaacgccttctaaaagctactggaatagtcgttgtccctggttctggctttagacaggtacctggaacatggcatttcaggtgcactatacttccccaagaggataagattccagcgattgttgatcgtctaactgcgttccaccagagcttcatggacgagttccgcgactaa

SEQ ID NO: 8 - protein, 545 aa, Arabidopsis thaliana
MRRFVIGQAKNLIDQSRRRQLHHHKNLSFVSLIPPFSAPSDSSSRHLSSSSSSDMSASDSSSSLPVTLDTINPKVIKCEYAVRGEIVNIAQKLQEDLKTNKDAYPFDEIIYCNIGNPQSLGQQPITFFREVLALCSYTALLDESATHGLFRFSSDSIERAWKILDQIPGRATGAYSHSQGIKGLRDAIADGIEARDGFPADPNDIFMTDGASPGVHMMMQLLITSEKDGILCPIPQYPLYSASIALHGGTLVPYYLDEASGWGLEISELKKQLEDARSKGITVRALAVINPGNPTGQVLSEENQRDVVKFCKQEGLVLLADEVYQENVYVPDKKFHSFKKVARSMGYGEKDLALVSFQSVSKGYYGECGKRGGYMEVTGFTSDVREQIYKMASVNLCSNISGQILASLIMSPPKPGDDSYESYIAEKDGILSSLARRAKTLEEALNKLEGVTCNRAEGAMYLFPCLHLPQKAIAAAEAEKTAPDNFYCKRLLKATGIVVVPGSGFRQVPGTWHFRCTILPQEDKIPAIVDRLTAFHQSFMDEFRD

SEQ ID NO: 9 - DNA, 26 bp, Artificial
  PCR primer
ctctagaaccgaacgtgactctccag

SEQ ID NO: 10 - DNA, 25 bp, Artificial
  PCR primer
ccatgatctccggcatctcatcttc

SEQ ID NO: 11 - DNA, 26 bp, Artificial
  PCR primer
atcacaaatcaggcacaaggttagac

SEQ ID NO: 12 - DNA, 26 bp, Artificial
  PCR primer
ggagggaagaagtgagctagggattg

SEQ ID NO: 13 - DNA, 26 bp, Artificial
  PCR primer
cgctcatcctggtatatgttctgctg

SEQ ID NO: 14 - DNA, 20 bp, Artificial
  PCR primer
ataacgctgcggacatctac

SEQ ID NO: 15 - DNA, 24 bp, Artificial
  PCR primer
ttagacaagtatctttcggatgtg

SEQ ID NO: 16 - DNA, 25 bp, Artificial
  PCR primer
aacgctgcggacatctacatttttg

SEQ ID NO: 17 - DNA, 30 bp, Artificial
  PCR primer
gtgggttaattaagaattcagtacattaaa

SEQ ID NO: 18 - DNA, 25 bp, Artificial
  PCR primer
aagaaaatgccgatacttcattggc

SEQ ID NO: 19 - DNA, 25 bp, Artificial
  PCR primer
aagaaaatgccgatacttcattggc

SEQ ID NO: 20 - DNA, 20 bp, Artificial
  PCR primer
tagatccgaaactatcagtg

SEQ ID NO: 21 - DNA, 26 bp, Artificial
  PCR primer
acgtgactccctttaattctccgctc

SEQ ID NO: 22 - DNA, 25 bp, Artificial
  PCR primer
cctaacttttggtgtgatgatgctg

SEQ ID NO: 23 - DNA, 25 bp, Artificial
  PCR primer
ttcccttaattctccgctcatgatc

SEQ ID NO: 24 - DNA, 25 bp, Artificial
  PCR primer
ttcccttaattctccgctcatgatc

SEQ ID NO: 25 - DNA, 25 bp, Artificial
  PCR primer
ttcccttaattctccgctcatgatc

SEQ ID NO: 26 - DNA, 27 bp, Artificial
  PCR primer
gtttcacatcaacattgtggtcattgg

SEQ ID NO: 27 - DNA, 24 bp, Artificial
  PCR primer
gagtacttgggggtagtggcatcc

SEQ ID NO: 28 - DNA, 30 bp, Artificial
  PCR primer
caataacaatgcaaagttaagattcggatc

SEQ ID NO: 29 - DNA, 23 bp, Artificial
  PCR primer
gcttcttctcaaccatcgtcacc

SEQ ID NO: 30 - DNA, 24 bp, Artificial
  PCR primer
ttcttcttctgaacgactattgtg

SEQ ID NO: 31 - DNA, 23 bp, Artificial
  PCR primer
gaatagggcaaagagaaagagtg

SEQ ID NO: 32 - DNA, 23 bp, Artificial
  PCR primer
ggtaacattgtgctcagtggtgg

SEQ ID NO: 33 - DNA, 23 bp, Artificial
  PCR primer
ggtgcaacgaccttaatcttcat

SEQ ID NO: 34 - DNA, 1443 bp, Oryza sativa Japonica
  CDS (1)..(1440)
atgttcggcggcggcggcggcggcgggaggaagccgctggactacgaggagctgaacgagaacgtgaagaaggtgcagtacgcggtgcggggggagctgtacctgcgcgcctccgagctccagaaggagggcaagaagatcatcttcaccaacgtcggcaacccacacgcgctcggccagaagccgctcaccttcccccgccaggttgtggcgctgtgccaggcccccttcctgctcgatgatcccaacgtcggccttatcttccccgccgacgccatcgcgcgggccaagcactacctcgccatggcacccggtggactagcttacagtgattcccgaggtatccctggtattaggaaggaagtcgccgagttcatcgagaggcgtgatggttatccagatccagaacttatttacctcacagatggtgccagcaaaggtgtgatgcaaatgctgaataccattatcagaaatgagagagatgggattctggttcctgttccacaatacccgctttattctgctgccatttccctctttggtggttctctcgtgccatactacttagaagaagaggctaactggggacttgacttcgtcaatctccgacagactgtggcgtcagcgcggtcaaagggaatcactgttcgagcaatggtgattatcaacccaggaaaccctactggccaatgccttagtgaaggaaacataaaggaacttctcaaattctgcttccatgagaacttagttctgcttgcagatgaagtctatcaacagaacatttatcaagatgagcgcccatttataagtgctagaaaggttctgtttgacatgggtcctcctatgagcagggaagttcagctggtttctttccatactgtgtcaaaaggatattggggggagtgtggacaacgtggagggtattttgaaatgacaaatcttcctcccaagacagtagacgagatctacaaggttgcatcaatcgcactcagtccaaatgttcctgggcagatctttatgggtttaatggttaaccctcctaagcctggagatatctcttatctgaagttttctgctgaaaagtctatcctcgagtctttgaggaggagagcacgcctgatgacagatggtttcaatagttgccgaaatgttgtctgcaatttcacagaagctatgtactctttcccccaaatacgcttaccaccaaaagctatagatgcagccaaaagggctggcaaagcggccgatgttttctactgcctcaagcttcttgaagcaactggaatatccactgttccagggtcaggtttcggacaaaaagaagtgttccacctgaggacgaccatcctgccagctgaggaggacatgcctgccatcatgaccagcttcaagaagttcaacgacactttcatggatcagtacgatggctactccaggatgtga

SEQ ID NO: 35 - protein, 480 aa, Oryza sativa Japonica
MFGGGGGGGRKPLDYEELNENVKKVQYAVRGELYLRASELQKEGKKIIFTNVGNPHALGQKPLTFPRQVVALCQAPFLLDDPNVGLIFPADAIARAKHYLAMAPGGLAYSDSRGIPGIRKEVAEFIERRDGYPDPELIYLTDGASKGVMQMLNTIIRNERDGILVPVPQYPLYSAAISLFGGSLVPYYLEEEANWGLDFVNLRQTVASARSKGITVRAMVIINPGNPTGQCLSEGNIKELLKFCFHENLVLLADEVYQQNIYQDERPFISARKVLFDMGPPMSREVQLVSFHTVSKGYWGECGQRGGYFEMTNLPPKTVDEIYKVASIALSPNVPGQIFMGLMVNPPKPGDISYLKFSAEKSILESLRRRARLMTDGFNSCRNVVCNFTEAMYSFPQIRLPPKAIDAAKRAGKAADVFYCLKLLEATGISTVPGSGFGQKEVFHLRTTILPAEEDMPAIMTSFKKFNDTFMDQYDGYSRM

SEQ ID NO: 36 - DNA, 1455 bp, Oryza sativa Indica
  CDS (1)..(1452)
atgttcggcggcggcggcggcgggaggaagccgctggactacgaggagctgaacgagaacgtgaagaaggtgcagtacgcggtgcggggggagctgtacctgcgcgcctccgagctccagaaggagggcaagaagatcatcttcaccaacgtcggcaacccacacgcgctcggccagaagccgctcaccttcccccgccaggttgtggcgctgtgccaggcccccttcctgctcgatgatcccaacgtcggccttatcttccccgccgacgccatcgcgcgggccaagcactacctcgccatggcacccggtggactaggtgcttacagtgattcccgaggtatccctggtattaggaaggaagtcgccgagttcatcgagaggcgtgatggttatccaagtgatccagaacttatttacctcacagatggtgccagcaaaggtgtgatgcaaatgctgaataccattatcagaaatgagagagatgggattctggttcctgttccacaatacccgctttattctgctgccatttccccctttggtggttctctcgtgccatactacttagaagaagaggctaactggggacttgacttcgtcaatctccgacagactgtggcgtcagcgcggtcaaagggaatcactgttcgagcaatggtgattatcaacccaggaaaccctactggccaatgccttagtgaaggaaacataaaggaacttctcaaattctgcttccatgagaacttagttctgcttgcagatgaagtctatcaacagaacatttatcaagatgagcgcccatttataagtgctagaaaggttctgtttgacatgggtcctcctatgagcagggaagttcagctggtttctttccatactgtgtcaaaaggatattggggggagtgtggacaacgtggagggtattttgaaatgacaaatcttcctcccaagacagtagacgagatctacaaggttgcatcaatcgcactcagtccaaatgttcctgggcagatctttatgggtttaatggttaaccctcctaagcctggagatatctcttatctgaagttttctgctgaaagcaagtctatcctcgagtctttgaggaggagagcacgcctgatgacagatggtttcaatagttgccgaaatgttgtctgcaatttcacagaaggagctatgtactctttcccccaaatacgcttaccaccaaaagctatagatgcagccaaaagggctggcaaagcggccgatgttttctactgcctcaagcttcttgaagcaactggaatatccactgttccagggtcaggtttcggacaaaaagaaggggtgttccacctgaggacgaccatcctgccagctgaggaggacatgcctgccatcatgaccagcttcaagaagttcaacgacactttcatggatcagtacgatggctactccaggatgtga

SEQ ID NO: 37 - protein, 484 aa, Oryza sativa Indica
MFGGGGGGRKPLDYEELNENVKKVQYAVRGELYLRASELQKEGKKIIFTNVGNPHALGQKPLTFPRQVVALCQAPFLLDDPNVGLIFPADAIARAKHYLAMAPGGLGAYSDSRGIPGIRKEVAEFIERRDGYPSDPELIYLTDGASKGVMQMLNTIIRNERDGILVPVPQYPLYSAAISPFGGSLVPYYLEEEANWGLDFVNLRQTVASARSKGITVRAMVIINPGNPTGQCLSEGNIKELLKFCFHENLVLLADEVYQQNIYQDERPFISARKVLFDMGPPMSREVQLVSFHTVSKGYWGECGQRGGYFEMTNLPPKTVDEIYKVASIALSPNVPGQIFMGLMVNPPKPGDISYLKFSAESKSILESLRRRARLMTDGFNSCRNVVCNFTEGAMYSFPQIRLPPKAIDAAKRAGKAADVFYCLKLLEATGISTVPGSGFGQKEGVFHLRTTILPAEEDMPAIMTSFKKFNDTFMDQYDGYSRM

SEQ ID NO: 38 - DNA, 30 bp, Artificial
  PCR primer
gcggatccatggctctcaaggcattagact

SEQ ID NO: 39 - DNA, 25 bp, Artificial
  PCR primer
gccgagctctcacattttcgaataa

SEQ ID NO: 40 - DNA, 20 bp, Artificial
  PCR primer
tgaaagcaaggggattcttg

SEQ ID NO: 41 - DNA, 20 bp, Artificial
  PCR primer
gacgtttttgcagctgttga

(19) United States
(12) Patent Application Publication
(10) Pub. No.: US 2005/0188435 A1
(43) Pub. Date: Aug. 25, 2005

(54) Method of increasing the GGT activity in plants, plants with increased GGT activity, and a method of producing such plants

(76) Inventors: Daisuke Igarashi, Kawasaki-shi (JP); Chieko Ohsumi, Kawasaki-shi (JP)

Correspondence Address: CERMAK & KENEALY LLP;ACS LLC, 515 EAST BRADDOCK ROAD, SUITE B, ALEXANDRIA, VA, 22314, US

(21) Appl. No.: 11/052,106

(22) Filed: Feb. 8, 2005

Related U.S. Application Data
(63) Continuation of application No. PCT/JP03/09946, filed on Aug. 5, 2003.

(30) Foreign Application Priority Data
Aug. 9, 2002 (JP) 2002-232562
Jul. 9, 2003 (JP) 2003-194431

(51) Int. Cl. A01H 1/00; C12N 15/82; C12Q 1/68

(52) U.S. Cl. 800/278; 435/468; 435/6

(57) ABSTRACT

The present invention describes a novel plant having increased activity of glutamate glyoxylate aminotransferase (GGT), a method of producing the plant, a seed of the plant, and a plant having an increased amino acid content. The present invention more specifically provides a plant which is improved in the content of at least one of the amino acid selected from the group consisting of serine, arginine, glutamine, and asparagine, and a method of producing the plant, as well as the seed of the plant. A plant having increased GGT activity is produced by mutagenesis, introduction of a nucleic acid molecule and the like. A genetic construction being capable of enhancing the expression of a GGT gene, particularly a genetic construct being capable of expressing a GGT gene and/or increasing the level of an endogenous gene having GGT activity is introduced into a plant.

This application claims priority as a continuation under 35 U.S.C. §120 to PCT/JP2003/009946 filed on Aug. 5, 2003.

### BACKGROUND OF THE INVENTION

1. Field of the Invention

The present invention relates to plants which have increased activity of glutamate glyoxylate aminotransferase (GGT). The present invention also relates to methods of utilizing glutamate glyoxylate aminotransferase (GGT) and/or a gene encoding GGT. The present invention also relates to methods of increasing the amino acid content of a plant and/or the seeds thereof, and more particularly, to methods of increasing the content of serine (Ser), arginine (Arg), glutamine (Gln), and/or asparagine (Asn). The invention further relates to to plants having increased amino acid content, particularly, plants having increased content of serine (Ser), arginine (Arg), glutamine (Gln), and asparagine (Asn), the plants and/or the seeds thereof, and to a method of producing such plants. Furthermore, the present invention relates to the use of the plants and/or the seeds thereof obtained according to the present invention for producing foods or feeds, and the present invention also relates to foods or feeds containing such plants and/or their seeds.

2. Brief Description of the Related Art

During photorespiration, glycolate produced by the oxygenase activity of RuBisco is metabolized. It has been reported that glycolate is metabolized to glyoxylate by glycolate oxygenase in peroxisomes, and that this glyoxylate is further metabolized by at least two glyoxylate aminotransferases (Somerville: PNAS 77: 2684-2687, 1980). Although a peroxisomal glyoxylate aminotransferase gene has not been previously identified, Liepman et al. recently reported an alanine: glutamate glyoxylate aminotransferase localized in the peroxisomes which functions in the photorespiratory system of Arabidopsis thaliana (Plant J. 25: 487-498). However, the glutamate glyoxylate aminotransferase gene was still unknown. In addition, the role that glutamate glyoxylate aminotransferase plays in various plant characteristics has not been clarified, particularly regarding characteristics such as amino acid content, including glutamate, the increase and decrease in total amino acid content, photosynthetic capacity, and stress tolerance. Moreover, the possibility to be able to improve plant characteristics by manipulating proteins with glutamate glyoxylate aminotransferase activity, or the gene encoding for such proteins, particularly a possibility to actually increase the total amino acid content and/or the content of specified amino acids in a plant or its seeds, has never been suggested in previous reports.

### SUMMARY OF THE INVENTION

An object of the present invention is to provide a plant which has increased glutamate glyoxylate aminotransferase (GGT) activity, a method of preparing the plant, and the seeds thereof.

Another object of the present invention is to provide a plant which has increased amino acid content, particularly those having increased content of serine (Ser), arginine (Arg), glutamine (Gln), and/or asparagine (Asn), as compared with a wild-type plant of the same species and cultivated under the same conditions, a method of preparing such plants, and the seeds of such plants.

Another object of the present invention is to provide methods of utilizing GGT and the genes encoding GGT.

More specifically, an object of the present invention is to provide a method of utilizing GGT and the gene encoding GGT to increase the amino acid content of a plant.

In addition, another object of the present invention is to provide feed and/or food containing plants and/or their seeds having increased amino acid content, particularly those having increased content of one or more amino acids selected from the group consisting of Ser, Arg, Gln, and Asn, and the use of such plants and/or their seeds for manufacturing of feed or food.

Moreover, a further object of the present invention is to provide a method of producing plant extracts containing one or more amino acids selected particularly from the group consisting of Ser, Arg, Gln, and Asn from the plants and/or their seeds having increased content of the above-mentioned amino acids, and to provide the use of such plants and/or their seeds for producing amino acids, particularly one or more amino acids selected from the group consisting of Ser, Arg, Gln, and Asn.

In addition, another object of the present invention is to provide plants and/or their seeds obtained according to the present invention as starting materials for the production of other substances for which amino acids are typically used as starting materials.

It is a further object of the present invention to provide a plant in which the transcription of a gene having GGT activity is increased as compared with a wild-type plant of the same species.

It is a further object of the present invention to provide a method of increasing the content of amino acids in plants, particularly the content of one or more amino acids selected from the group consisting of serine, arginine, glutamine and asparagine in the plant, comprising increasing the GGT activity.

It is a further object of the present invention to provide a transgenic plant into which a gene construct capable of increasing the expression of GGT gene is introduced, particularly a gene construct capable of expressing the GGT gene and/or a gene construct capable of increasing the expression of genes with endogenous GGT activity, wherein the GGT activity of the transgenic plants is increased as compared with the wild-type plants of the same species or the corresponding non-transformed plants which were cultivated under the same conditions.

It is a further object of the present invention to provide a method of increasing the GGT activity of plants comprising introducing a gene construct capable of increasing the expression of the GGT gene, particularly, a gene construct capable of expressing the GGT gene and/or a gene construct capable of increasing the transcription of genes having endogenous GGT activity.

The present invention is also a method of producing plants which have an increased GGT activity comprising geminating the plants having increased GGT activity as compared with a wild-type plant of the same species or the plant seeds having increased GGT activity as compared with the corresponding non-transformed plants, or regenerating plant bodies from the above-mentioned plants or transformed plant cells, or by proliferating the plants or transgenic plants by vegetative proliferation.

Particularly, according to the present invention, the GGT activity specifically means the GGT activity in peroxisomes.

### BRIEF DESCRIPTION OF THE DRAWINGS

FIG. 1 illustrates a diagram of the photorespiration pathway in higher plants. The large arrow indicates the reaction catalyzed by glutamate glyoxylate aminotransferase.

FIG. 2 shows a comparison of the amino acid sequences of glutamate glyoxylate aminotransferase from Arabidopsis thaliana. Identical amino acids are indicated by asterisk.

FIG. 3 shows the structure of the glutamate glyoxylate aminotransferase gene from Arabidopsis thaliana and the location where the gene is inserted in pBI101. Exons are shown as black boxes. The genomic 5089 bp region was amplified by PCR and cloned into pBI101 (-GUS/-NOS-ter) using a BmaHI site on the genome and a Hind III site on the primer. Using this vector, the clone was introduced into a GGT1 gene knockout line (ggt1-1) by way of Agrobacterium-mediated transformation.

FIG. 4 shows a comparison between the growth of the control strain and the GGT1 introduced strain (ggt1-1/GGT1). The weight was measured of the above-ground part of 95 individual seedlings from a wild-type non-transformed strain (Control) and the GGT1-introduced strain (ggt1-1/GGT1), both cultivated for 2 weeks under ordinary culture conditions.

FIG. 5 is a graph comparing the GGT1 mRNA levels of the control strain and the GGT1-introduced strain.

FIG. 6 is a graph showing a comparison of the GGT enzyme activity levels of the control strain and the GGT1-introduced strain.

FIG. 7 shows the results of measuring the amino acid content in the seedlings grown for 2 weeks on PNS medium under light conditions of 70 μmol m⁻² s⁻¹. (A): individual amino acid content in the seedling (nmol/mg FW), and (B): total amino acid content in the seedling (nmol/mg FW).

FIG. 8 shows the amino acid content of the rosette leaves of the plant body cultivated for 42 days on rock wool using PNS as a fertilizer under light conditions of 70 μmol m⁻² s⁻¹. (A): individual amino acid content (nmol/mg FW), (B): total amino acid content (nmol/mg FW).

FIG. 9 is a graph showing a comparison of the GGT1 mRNA levels of the GGT1-introduced strains and the control strain.

FIG. 10 is a graph showing a comparison of the GGT enzyme activity (A) and the HPR activity (B) of the GGT1-introduced strain and the control strain. The enzyme activity of the control plant was set as 1.

FIG. 11 shows the results of measuring the serine content of the seedlings cultivated for 2 weeks on PNS medium under light conditions of 70 μmol m⁻² s⁻¹.

FIG. 12 shows a comparison of GGT1 mRNA levels, GGT enzyme activity levels, and Ser content of a transgenic plant produced by introducing a construct for expressing GGT1 into a wild-type strain and a control strain. The resulting correlation coefficient and regression formula are shown. (A): the relative GGT enzyme activity vs. the relative GGT1 mRNA level, (B): the Ser content vs. the relative GGT1 mRNA level, and (C): the Ser content vs. the relative GGT enzyme activity.

FIG. 13 shows the results of measuring the amino acid content of the seedlings grown for 2 weeks on ½ MS medium under light conditions of 70 μmol m⁻² s⁻¹. (A): the individual amino acid content, and (B): the total amino acid content.

FIG. 14 shows the amino acid content of the seeds obtained from plant bodies cultivated under continuous lighting (about 200 μmol m⁻² s⁻¹) with the modified PNS fertilizer (5 mM KNO₃ was replaced by 2.5 mM NH₄NO₃)(n=4). (A): individual amino acid content, (B): arginine content, and (C) total amino acid content, each in nmol/mg FW.

FIG. 15 shows the results of another experiment performed under the same conditions as that shown in FIG. 14 (n=2). (A): individual amino acid content, (B): arginine content, (C): total amino acid content, each in nmol/mg FW.

FIG. 16 shows the amino acid homology between Arabidopsis thaliana GGT and the proteins which are suspected to be rice (Oryza sativa) GGT protein. GGT1: Arabidopsis thaliana GGT1, Japonica_GGT: suspected GGT protein from Oryza sativa japonica, and Indica_GGT: suspected GGT protein from Oryza sativa indica. Identical amino acids are indicated by an asterisk.

FIG. 17 shows the results of measuring the amino acid content of the daytime leaves of primary transgenic rice plants into which the Arabidopsis-derived GGT gene was introduced. The numerical values given are relative to the total amino acid content. Individual amino acid content, relative to the total amino acid content, of about 10% were selected and shown in the figure.

### DETAILED DESCRIPTION OF THE PREFERRED EMBODIMENTS

As used herein, and in contrast to the “transgenic plants” defined herein, the term “non-transgenic plants” means “plants into which a genetic construct capable of increasing the expression of the GGT gene was not introduced”. Such plants include wild-type plants and plants into which a genetic construct other than the genetic construct capable of increasing the expression of the GGT gene has been introduced. In addition, “a genetic construct capable of increasing the expression of the GGT gene” includes a gene construct capable of expressing the GGT gene, for example, a gene construct containing the GGT gene which is functionally or operably linked to an appropriate promoter, and a genetic construct capable of increasing the transcription of the GGT gene, for example, a construct containing an enhancer. The term a “genetic construct” as used herein means any construct capable of being inherited by descendents in any form. Particularly, “genetic constructs” encompass nucleic acid molecules. When the genetic construct contains a gene, it may be specifically referred to as a “gene construct”. Therefore, for example, “a genetic construct” not only includes nucleic acid molecules containing a gene but also includes nucleic acid fragments containing a transcriptional activation element, an enhancer, or the like.

More specifically, the present invention describes plants in which the activity of GGT having homology of 60% or more in the amino acid sequence to the amino acid sequence in SEQ ID No. 2 or 4 is increased as compared with the wild-type plant of the same species cultivated under the same conditions.

In particular, the present invention describes plants having increased GGT activity as compared with a wild-type plant cultivated under the same conditions, wherein GGT has the amino acid sequence in SEQ ID No. 2 or 4.

Moreover, the present invention describes transgenic plants into which a genetic construct containing a nucleotide sequence capable of hybridizing with the polynucleotide in SEQ ID No. 1 or 3 under stringent conditions is introduced, wherein the GGT activity of the transgenic plants is increased as compared with the corresponding non-transformed plants cultivated under the same conditions.

In particular, the present invention describes a transgenic plant into which a genetic construct containing the nucleotide sequence in SEQ ID No. 1 or 3 is introduced, wherein the GGT activity of the transgenic plant is increased as compared with the corresponding non-transgenic plant cultivated under the same conditions.

Moreover, the present invention describes a method of increasing amino acid content, particularly, the content of Ser, Arg, Gln, and/or Asn in plants and/or their seeds, the method including preparing transgenic plants by introducing a gene construct capable of expressing GGT, wherein the gene construct is able to increase the GGT activity of the transgenic plants as compared with the corresponding non-transgenic plants cultivated under the same conditions, and to plants having increased total amino acid content, particularly plants and/or their seeds having increased content of Ser, Arg, Gln, and/or Asn.

The GGT activity of the plants of the present invention is increased preferably about 1.2-fold or more, more preferably about 3-fold, or more and most preferably about 5-fold or more, compared with the GGT activity level in the corresponding tissues of wild-type plants, or non-transgenic plants, cultivated under the same condition.

The objects of the present invention may be achieved by selecting or preparing plants in which the glutamate glyoxylate aminotransferase (GGT) activity is increased as compared to wild-type plants of the same species, or by selecting or preparing transgenic plants in which GGT activity is increased as compared with the corresponding non-transgenic plants.

For example, an object of the present invention may be achieved by increasing the expression of a glutamate glyoxylate aminotransferase gene (GGT gene) by introducing into a plant a genetic construct capable of increasing the expression of a gene encoding GGT. Such genetic constructs include those capable of expressing GGT, those capable of expressing a transcription activating factor or a nucleic acid fragment which functions to increase the transcription activity, and the like.

In one embodiment of the present invention, a transgenic plant is selected in which the expression of the gene coding for GGT is increased by introduction of a gene construct capable of expressing GGT, as compared with the corresponding non-transformed plant cultivated under the same conditions.

In another embodiment of the present invention, the expression of the GGT gene is increased by increasing the copy number of the GGT gene. In another embodiment of the invention, the transcription of GGT gene is increased by the expression, more preferably by the overexpression, of a transcriptional activator, and the GGT activity is increased as a consequence. In one embodiment of the invention, the transcription of the GGT gene is increased by introducing an enhancer, and the like including a cis-element having a transcription-activating function, and the GGT activity is increased as a consequence.

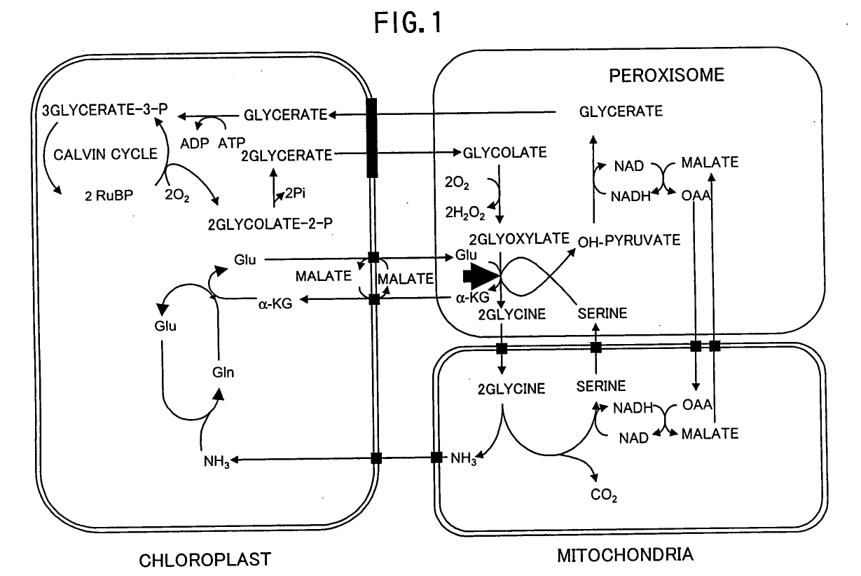

The term “glutamate glyoxylate aminotransferase” as used herein is the name for proteins having glutamate glyoxylate aminotransferase activity, namely, proteins possessing an activity which catalyzes the reaction: glyoxylate+glutamate→glycine+α-ketoglutarate (FIG. 1). In particular, such proteins include, for example, proteins having amino acid sequence homology of at least 60%, preferably about 70% or more and most preferably 90% or more, to the amino acid sequence in SEQ ID No. 2 or 4. This homology can be calculated by using programs well-known to those skilled in the art, such as FASTA, using standard parameters. For example, FASTA Versions 2.0, 3.0, 3.2 3.3, and the like are available with standard parameters from DNA • Data Bank of Japan (DDBJ/CIB) (http://www.ddbj.nig.ac.ip/Welcome-j.html), National Institute of Genetics.

Similarly, “the gene encoding GGT” or “the GGT gene” includes any gene encoding a protein having glutamate glyoxylate aminotransferase activity. In particular, such genes include those having the nucleotide sequence homology to the nucleotide sequence described in SEQ ID No. 1 or 3 of preferably 70% or more, and more preferably about 90% or more. This homology can also be calculated by using, for example, the FASTA and the like. The nucleic acid molecules having such a homology are also nucleic acid molecules that can be hybridized with the nucleic acid molecules having the sequence of SEQ ID No. 1 or 3 under stringent conditions. The proteins that are encoded by such genes include proteins possessing the amino acid sequences having addition, substitution and deletion of amino acid sequences in the amino acid sequence in SEQ ID No. 2 or 4.

The term “stringent conditions” as used herein means conditions under which a specific hybrid is formed but non-specific hybrids are not formed. It is difficult to numerically express this condition; however, the following conditions are exemplary: a condition in which a pair of highly homologous DNAs, for example, a pair of 70% or more homologous DNAs, hybridize to each other, but a pair of DNAs with lower homology do not hybridize, or a hybridization condition where the washing conditions of Southern hybridization are 50° C., 2×SSC and 0.1% SDS, preferably 1×SSC and 0.1% SDS, more preferably 0.1×SSC and 0.1% SDS. Although the genes that can hybridize under such conditions may include genes having stop codons or mutations at the active center, such genes can be easily eliminated by linking to a commercially available activity-expressing vector and by measuring the GGT enzyme activity conventionally.

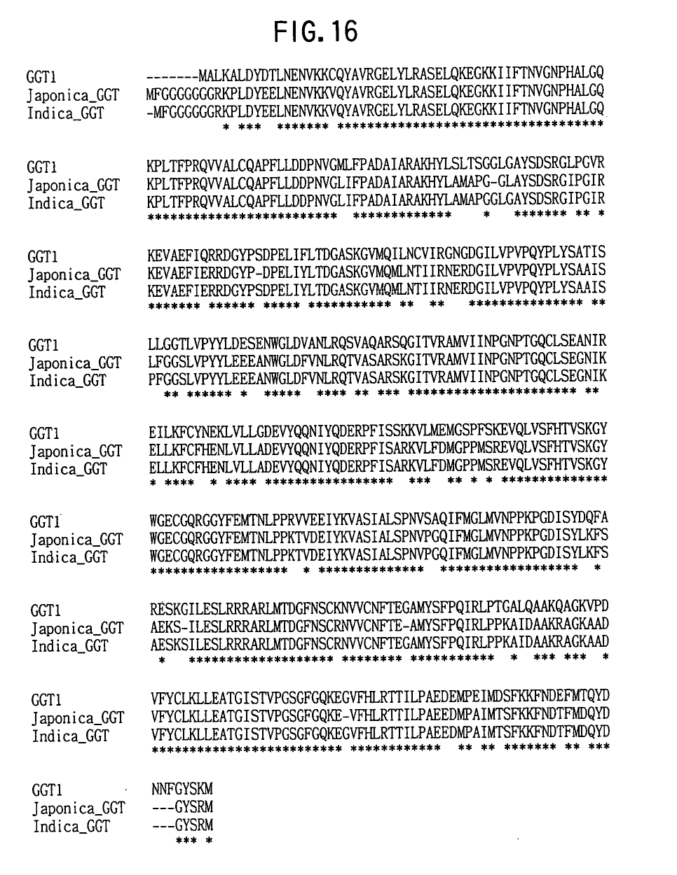

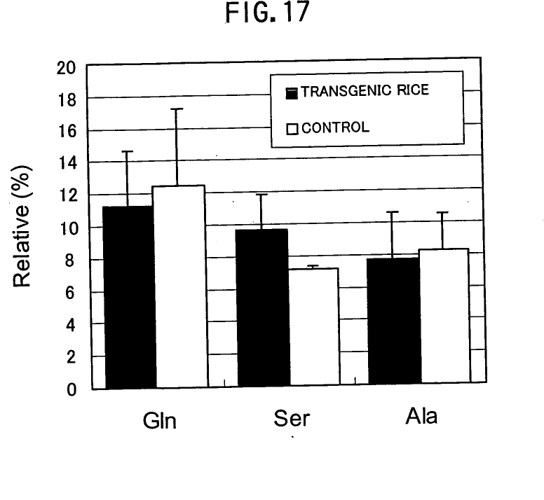

Thus, any genes or proteins are encompassed that have the gene sequence homology to SEQ ID No. 1 or 3, or the amino acid sequence homology to SEQ ID No. 2 or 4, and can be utilized as equivalents of these genes or proteins according to the present invention, for example, those derived from rice. As such examples, the nucleotide sequence of the suspected GGT gene of Oryza sativa japonica, and the amino acid sequence of the protein, which may be encoded by this gene, are described in SEQ ID Nos. 34 and 35, respectively, and, similarly, the gene sequence of the suspected GGT gene of Oryza sativa indica, and the amino acid sequence of the protein, which may be encoded by this gene, are described in SEQ ID Nos. 36 and 37, respectively. Homology of the amino acid sequence between Arabidopsis GGT1 and these rice proteins is shown in FIG. 16. It is clear that the homology at the amino acid sequence level between GGT1 and the proteins from japonica and indica corresponding to the GGT1 is very high.

In addition, the GGT genes which can be used in the present invention may be either isogenic genes derived from plants to be transformed, or the heterologous genes obtained from other sources.

The phrase “transgenic plant having an increased GGT activity compared with the corresponding non-transformed plants cultivated under the same conditions” as used herein means the transgenic plant of which total GGT activity due to both the inherent GGT gene of the corresponding non-transgenic plant and the GGT gene existing on the gene construct used for transformation is increased as compared with the GGT activity of the corresponding non-transformed plant cultivated under the same conditions, namely, a plant which belongs to the same species as said transgenic plant but was not transformed with a GGT gene-expressing construct. It was already mentioned that, in comparison with the transgenic plant into which a gene construct capable of expressing GGT was introduced, the term, “non-transgenic plant”, means the “the plant into which a gene construct capable of expressing GGT was not introduced”.

The GGT activity may be increased either at the transcription level, translation level, or post-translational modification level. For example, the GGT activity can be increased by introducing a gene construct capable of expressing GGT, and by controlling the upstream elements involved in the control of the GGT activity and/or transcription amount, such as the GGT expression regulatory element, translation regulatory element, and post-translational regulatory element. More specifically, for example, the GGT activity can be increased by introducing a gene construct capable of expressing GGT in particular, by increasing the copy number of endogenous GGT gene, by introducing a transcriptional activator, by introducing an enhancer elevating the transcription activity of the endogenous GGT gene, or the like. These methods are well-known to those skilled in the art. For example, it is known that when the DREB1A gene is expressed under the control of the promoter of the rd29A gene (stress-induced promoter), the expression of the target gene DREB1 greatly increases in response to stress, as compared with wild-type plants (Nature Biotechnology, 17 287-, 1999). It has also been reported that a target gene can be identified by randomly inserting an enhancer for activating transcription and selecting individuals having a characteristic trait among them (Plant J., 34, 741-750, 2003; Plant Physiol., 129, 1544-1446, 2002).

According to the present invention, the GGT activity of the transgenic plant of the present invention is increased preferably about 1.2-fold or more, more preferably about 3-fold or more, and most preferably about 5-fold or more, as compared with the GGT activity of the corresponding tissues of non-transgenic plant cultivated under the same conditions.

In addition, even at the mRNA level, the GGT mRNA level of the transgenic plant of the present invention increases up to preferably about 2-fold or more, more preferably about 5-fold or more, and most preferably about 30-fold or more, as compared with the GGT mRNA level of the corresponding tissues of the non-transformed plant cultivated under the same conditions. There is a strong positive correlation between the GGT activity and the mRNA level in the plant of the present invention and the plants obtained according to the present invention.

A plant having increased GGT activity only in a specific tissues, for example a plant wherein the GGT activity is increased only in stems, including tubers, leaves, or in flowers, and a method of producing such a plant are also included within the scope of the present invention. Therefore, even if the increase in total amino acid content, or the increase in the amino acid content of at least one of Ser, Arg, Gln, and/or Asn is found only in a part of the plant, the plants or the methods are also within the scope of the present invention.

According to the present invention, the increase in the GGT activity preferably occurs in a peroxisome, particularly in a peroxisome of photosynthesis tissue. The photosynthesis tissue may be tissue which photosynthesizes under conventional culture conditions or cultivation conditions including a leaf, a stem, a silique, and the like.

The GGT genes used as the target in the present invention can also be obtained from various plants. For example, DNA base sequence information of GGT genes can be obtained by retrieving it from a database using “alanine aminotransferase” as a keyword. According to the sequence information, the full-length cDNA can be obtained by using RT-PCR, 5′-RACE, or 3′-RACE. It is also possible to obtain the cDNA by screening cDNA libraries by hybridization with a suitable probe according to the known sequence information. The probes used for the screening can be prepared based on the amino acid or nucleotide sequence of GGT.

According to the present invention, the GGT gene is preferably localized in peroxisomes, particularly peroxisomes in the photosynthesis tissues as described above. The localization of GGT in the peroxisomes can be determined from the presence of N-terminal or C terminal sequences characteristic to the proteins localized in the peroxisomes. Such sequences include, for example Arg-(Leu/Gln/Ile)-X5-His-Leu and similar sequences as the N-terminal sequences, and (Ser/Ala)-(Arg/Lys)-(Ile/Leu/Met) and similar sequences as the C-terminal sequences. A protein having the GGT activity may be connected to such N-terminal or C-terminal sequence which is characteristic of a peroxisome-localized protein. Additionally, to confirm the localization the resulting GGT gene may be fused to a reporter gene such as GFP or GUS while maintaining the localization to peroxisomes and the fused gene may be expressed in a cell and tested. Alternatively, a GGT having a tag may be expressed and detected by a specific antibody to confirm the localization.

The gene constructs for increasing expression of the GGT gene according to the present invention may be generated by methods well-known to those skilled in the art. The promoter for expressing the GGT gene may be any promoter which can function in a plant. For example, a gene construct where GGT expression is driven by a cauliflower mosaic virus (CaMV) 35S promoter (EMBO J. 6: 3901-3907, 1987), a maize ubiquitin promoter (Plant Mol. Biol. 18: 675-689, 1992), an actin promoter, a tubulin promoter, and the like, are encompassed. The high expression promoters are particularly preferable. The terminators may also be those which can function in a plant cell. For example, the terminator from CaMV or the terminator from nopaline synthase gene can be used. The GGT expression unit which may exist in a plant genome may be also used. Molecular biology techniques including procedures for designing nucleic acid constructs, isolating them, and determining the sequences thereof may be found in the literature such as Sambrook et al., Molecular cloning-Laboratory manual, Edition 2, Cold Spring Harbor Laboratory Press. To prepare the nucleic acid constructs usable in the present invention, gene amplification procedures including a PCR method may be required in some cases. As for such procedures, for example, F. M. Ausubel et al. (eds), Current Protocols in Molecular Biology, John Wiley & Sons, Inc. (1994) can be referenced.

The method of introducing the nucleic acid construct in the above-described embodiment is not particularly limited. Any method for introducing genes into plant cells or into plant bodies, known by those skilled in the art, can be selected according to the chosen host. For example, the Agrobacterium-mediated gene introduction method, the electroporation method, or a particle gun can be employed. When Agrobacterium is used, the sequence to be introduced is preferably inserted between the left and right T-DNA border sequences. The suitable design and construction of the transformation vectors based on T-DNA are well known in the art. Furthermore, the conditions required for infection of a specified plant with agrobacteria harboring such a nucleic acid construct are also well known in the art. As for such techniques and conditions, Cell Technology, additional volume, “Model Shokubutsu no Jikken Protocol; Ine, Shiroinunazuna Hen (Experiment Protocol for Model Plants; Edition of Rice Plants and Arabidopsis thaliana) published by Shujunsha (1996) can be referenced.

Although the plant species to be subjected to gene manipulation are not particularly limited, they are preferably those which can be easily cultivated and transformed, and have established regeneration systems. In addition to the plants having the above-described characteristic properties, plant species for which large-scale cultivation techniques have been established and which have a high utility value as food, are preferred in the present invention. Those plants include, in addition to Arabidopsis thaliana as the model plant, rice, maize, wheat, sugar beet, cassava, spinach, cabbages, lettuce, salad, celery, cucumber, tomato, broad bean, soybean, adzuki bean, kidney bean, and pea. These plants may be naturally occurring plants or those that have already received a genetic modification, such as plants where expression of the intrinsic natural GGT gene is increased. The plants that have received any genetic modification may be selected from an existing library, for example from an existing high-expression library.

Then the genetically manipulated plant cells and the like thus obtained are subjected to the selection of transformants. The selection may also be based on the expression of marker genes present on the nucleic acid construct used for the transformation. For example, when the marker genes are drug resistant genes, the selection can be conducted by culturing or growing the manipulated plant cells on a culture medium containing a suitable concentration of an antibiotic or a herbicide. When the marker gene is, for example a β-glucuronidase gene or a luciferase gene and the like, the transformants can be selected by screening for the activity. From the thus identified transformants such as protoplasts, calli, and explants, the plant bodies can be regenerated. Known regeneration methods for each host plant may be employed. The plants thus obtained can be cultured by an ordinary method or, in other words, under the same conditions as those for the untransformed plants or under conditions suitable for the respective transformants. For the identification of the transgenic plants containing the nucleic acid constructs of the present invention, various molecular biological methods can be employed in addition to the above-described marker gene selection method. Southern hybridization, PCR, Northern hybridization, and RT-PCR and the like may be used to confirm the insertion of GGT gene into the genome, to identify the location of the insertion, to confirm the inserted copy numbers, and the like.

The amount of GGT protein, the GGT activity, and the amount of mRNA of GGT may be estimated in the resulting transgenic plants. For example, the amount of the protein can be determined by a Western blotting method or the like, and the amount of the mRNA can be determined by a Northern blotting method, a quantitative RT-TCR method, or the like. GGT activity can be determined by an ordinary method (Plant Physiol. 99: 1520-1525). For example, GGT activity in a photosynthetic tissue can be determined by freezing the tissue, such as leaves, with liquid nitrogen, pulverizing the frozen tissue, suspending the obtained powder in a suitable extraction buffer such as a buffer containing 100 mM Tris-HCl (pH 7.3) and 10 mM DTT, ultra-filtrating the obtained suspension, and subjecting the obtained specimen to the above-described determination method (Plant Physiol. 99: 1520-1525). GGT activity localized in the peroxisome can be determined by isolating the peroxisomes by an ordinary method (Plant Physiol. 43: 705-713, J. Biol. Chem. 243: 5179-5184, Plant Physiol. 49: 249-251 or the like) and then determining the activity by the above-described method. These methods are well-known in the art.

According to the present inventions, the GGT activity of the transgenic plants increases more than about 1.2-fold, preferably more than about 3-fold, most preferably more than about 5-fold as compared with the GGT activity in the corresponding tissue of the corresponding non-transformed plants which are cultivated under the same conditions.

The amino acid content may be determined in the resulting plants. The amino acid content can be determined by, for example, pulverizing the plant body or a part thereof and examining the extract with a conventional amino acid analyzer. For example, amino acids can be extracted by adding 500 μl of 80% ethanol to a sample (a plant body or a part thereof), pulverizing the sample with a cell blender MM 300 (QIAGEN) and treating the obtained product at 80° C. for 10 minutes. The product is centrifuged and then subjected to vacuum concentration. The remaining sample is dissolved in 0.02 N HCl to obtain an analysis sample. The sample is passed through a 0.22 μm filter to remove impurities. For the amino acid analysis, amino acid content can be determined with an amino acid analyzer LS-8800 (HITACHI). The amino acid content in a plant may be quantified by measuring the total amount of amino acids, the amount of at least one of serine (Ser) and/or arginine (Arg), or rate increase of the amount of total amino acids, at least one of Ser, Arg, Gln, and Asn as an indicator, and is optionally processed statistically, in a particular tissue, preferably a photosynthesis tissue such as a leaf compared to the control plant grown under the same conditions. When the increase in at least one of these indices is statistically significant, the total amino acid content or the content of at least one of Ser, Arg, Gln, and Asn is significantly increased as compared with that of the control plant, respectively, depending on the results.

A plant where the expression of the GGT gene is increased may be obtained from a plant library where an enhancer or a T-DNA tag has been randomly inserted.

Furthermore, a plant where the expression of the GGT gene is increased may be obtained without using direct molecular biology techniques such as those described above. Namely, a plant where GGT gene expression is enhanced and GGT activity is increased can be obtained via the action of a known plant mutagen, and selecting the plant using the aforementioned properties. The substances for inducing a mutation and the methods of introducing a mutation into a plant are well known to those skilled in the art. For example, EMS, methylnitrosourea, γ-ray, ion beam, X-radiation may be used as a mutagen.

According to the present invention, a plant can be obtained having increased amino acid content, particularly a plant where the content of at least one of Ser, Arg, Gln, and Asn is increased. Specifically, according to the present invention, a mature plant can be obtained wherein the amino acid content of the plant is preferably increased about 1.5-fold, more preferably about 4-fold as compared with the corresponding non-transformed plant or the wild-type plant which is cultivated under the same conditions. Particularly, the Ser content may increase more than about 2-fold, preferably more than about 3-fold, particularly preferably more than 20-fold as compared with the wild-type plant of the same species or the corresponding non-transformed plant. Regarding Arg, Gln, Asn content, a more than 1.5-fold increase, preferably more than 3-fold increase, most preferably more than 5-fold increase is achieved. Especially, a more than 5-fold increase is achieved for Asn and Arg.

Additionally, Ser content can be particularly increased by cultivating the plants of the present invention by limiting the nitrogen fertility to nitrate nitrogen. On the other hand, Asn, Gln, and Arg content, as well as Ser content, can be increased by incorporating ammonia nitrogen in the nitrogen fertilizer. Thus, the amino acid content of the plants of the present invention may be controlled by changing the cultivation conditions, and particularly by changing the nature of the nitrogen fertilizer.

Once the plant having increased amino acid content is identified, it is possible to examine whether the characteristics thereof can be stably kept genetically or not. For this purpose, plants may be cultivated under ordinary light conditions, the seeds thereof may be taken, and the phenotypes and the segregation of the descendants thereof may be analyzed. For the transformants, the presence or absence of the introduced nucleic acid constructs, the position thereof, and the expression thereof in the progenies may be analyzed in the same manner as that of the primary transformants. When the plants are obtained without using direct gene introduction, the presence or absence of the mutations and their location can also be similarly analyzed.

The plants having increased amino acid content are either heterozygous or homozygous regarding the sequence derived from the nucleic acid constructs integrated into the genomes or as for the mutated or disrupted genes. If necessary, either heterozygotes or homozygotes can be obtained by, for example, cross-fertilization. The sequences derived from the nucleic acid constructs which have been integrated into the genomes segregate according to Mendel's law in the descendants. Therefore, for the objects of the present invention, it is preferable to use homozygous plants due to the stability of the characters. The plants of the present invention can be grown under ordinary cultivation conditions.

The plants according to the present invention may be produced and/or propagated by regenerating the plant bodies from the cells or the parts of the plants having increased GGT activity or those having increased amino acids content as described above. The plants having features of the plants according to the present invention may be regenerated by culturing the cells or the tissues of the plants of the present invention on a medium where MS basal medium is supplemented with appropriate hormones, optionally through the formation of embryogenesis or cell aggregation such as callus formation. Techniques for regenerating a plant body from plant cells or from parts of plants are well known to those skilled in the art. If the plants according to the present invention having increased GGT activity or having increased amino acid content as described above are capable of seed propagation, the plants according to the present invention having the aforementioned features may be obtained by collecting seeds, preferably heterozygous seeds, from the plants and seeding them according to conventional procedures, such as simply seeding them in an appropriate soil.

In the production of the seeds of the present invention, it is particularly preferred to cultivate the homozygous plants and harvest the seeds thereof. The homozygous plants may be selected by repeating the cultivation of the generations until the interested phenotypes do not segregate or, in other words, the homozygous plants can be selected by selecting the lines exhibiting the interested phenotype in all the progenies thereof. The homozygotes can be selected by PCR or Southern analysis. By determining amino acid content of the plant by a method such as the above-described method, the seeds of the present invention may be confirmed to have a higher amino acid content than the seeds of the corresponding wild-type plant cultivated under the same conditions, especially as to the content of at least one of Ser, Arg, Gln, and Asn.

Additionally, if the plants according to the present invention are capable of vegetative propagation, the plants having the features of the plants of the present inventions can be directly propagated from parts of the plants. These propagation procedures are well known to those skilled in the art (For example, “Engei-Daihyakka 10 Saibai no Houhou”, 1980, Koudansha may be referenced). Such vegetative propagation procedures include, but are not limited to, procedures using tuberous roots or tubers such as those used for the potato family or carrot, and those using cuttage or graftage of plants. The properties of the plants produced and/or propagated as such can be estimated, particularly the amino acid content as described above.

The plants and seeds of the present invention are usable as foods and food materials in the same manner as the corresponding wild-type plants. Therefore, the plants and seeds of the present invention are directly usable as foods, or after cooking or processing by an ordinary method, they can be also used as feed products.

To obtain a plant extract containing amino acids, particularly at least one of Ser, Arg, Gln, and Asn from the plants having increased amino acids content, particularly from the plants where at least one of Ser, Arg, Gln, and Asn is increased, conventionally known methods for extracting amino acid fractions from plants, especially those for extracting fractions containing at least one of Ser, Arg, Gln, and Asn can be used. For purification of any one of Ser, Arg, Gln or Asn from the extract containing at least one of these amino acids, numerous methods known to those skilled in the art can be used, including various chromatography methods.

The following non-limiting examples will further illustrate the methods for obtaining the plants of the present invention by using a model plant Arabidopsis thaliana and rice plants as a starting material, and also illustrate the features of resulting plants and seeds. It will be apparent for those skilled in the art that the plants of the present invention, their seeds and the methods of the present invention are not limited to the particular plants, Arabidopsis thaliana and Oryza sativa (rice).

According to the disclosure of the present specification, it will be apparent to those skilled in the art that the GGT gene may be used as a marker gene in the production of a transgenic plant. For example, the GGT gene may be used for inducing resistance against substances which may specifically inhibit GGT or inducing stress-resistance to screen transgenic plants under the existence of such substances or stresses.

### EXAMPLES

All plant cultivation was performed under the following conditions.

PNS (Mol. Gen. Genet. 204: 430-434) or MS (Physiol Plant 15: 473-479) inorganic salts containing 1% (w/v) sucrose, 0.05% (w/v) MES [2-(N-morpholino) ethanesulfonic acid] and 0.8% (w/v) agar were used as the basal medium for plates. During cultivation on rock wool, only PNS inorganic salts were used as a source of nutrients.

The GGT-knockout Arabidopsis thaliana strain, which had been previously obtained, was used in the transformation experiments as a model. The method of preparing the GGT-knockout strains is shown in Reference Examples 1 and 2.

### Reference Example 1

### Preparation of GGT-knockout Arabidopsis thaliana Lines

(1) Preparation of Primers for Screening GGT-knockout Lines

Since the GGT gene is also the AlaAT gene, the GGT gene was obtained based on the information about the alanine aminotransferase (AlaAT) gene of Arabidopsis thaliana.

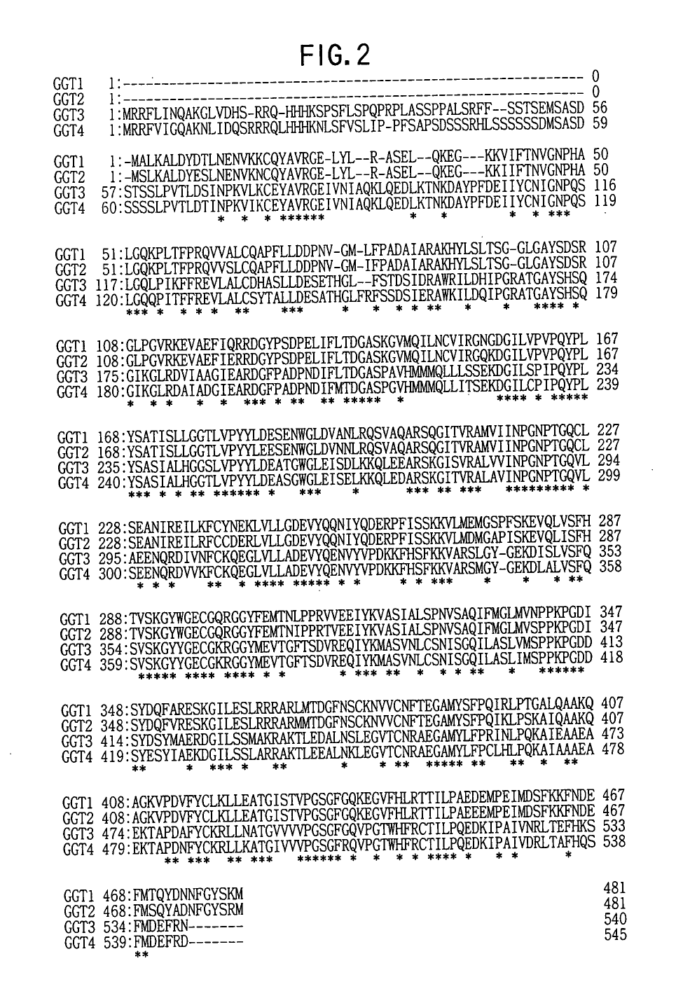

To prepare primers, the copy number and the sequence of AlaAT are estimated from the data available on the Internet. Conducting a search using “Alanine aminotransferase” and “Arabidopsis” as key words, it was found that at least 4 copies of the genes, which were supposed to be alanine aminotransferase, were present on the genome. Genbank accession numbers of the respective genes were AC005292 (F26F24.16), AC011663 (F5A18.24), AC016529 (T10D10.20) and AC026479 (T13M22.3). The genes were named GGT1, GGT2, GGT3, and GGT4, respectively. The cDNA nucleotide sequences are shown in SEQ ID Nos: 1, 3, 5, and 7, respectively, and the dedicated amino acid sequences are shown in SEQ ID Nos: 2, 4, 6, and 8, respectively. The homology of GGT2, GGT3, or GGT4 with GGT1 is shown in Table 1. The comparison of the deduced amino acid sequences is shown in FIG. 2.

TABLE 1
% Homology between GGT1 and GGT2, GGT3, or GGT4

| | Homology in amino | Homology in cDNA |
|---|---|---|
| | acid sequence | nucleotide sequence |
| GGT2 | 92.93 | 75.68 |
| GGT3 | 44.71 | 46.72 |
| GGT4 | 44.67 | 48.06 |

According to the EST information, the expression of GGT1 was supposed to be highest among the 4 genes. PCR primers for screening the gene disrupted strains were prepared based on the GGT1 sequence (Table 2). These primers were designed according to the system provided by Kazusa DNA Laboratory.

TABLE 2
PCR primers for screening the gene disrupted strains

| | |
|---|---|
| Name | Sequence * |

| | | |
|---|---|---|
| AAT1 U | CTCTAGAACCGAACGTGACTCTCCAG | (SEQ ID NO:9) |
| AAT1 L | CCATGATCTCCGGCATCTCATCTTC | (SEQ ID NO:10) |
| AAT1 L2 | ATCACAAATCAGGCACAAGGTTAGAC | (SEQ ID NO:11) |
| AAT RTU | GGAGGGAAGAAGTGAGCTAGGGATTG | (SEQ ID NO:12) |
| AAT RTL | CGCTCATCCTGGTATAT GTTCTGCTG | (SEQ ID NO:13) |
| 00 L | ATAACGCTGCGGACATCTAC | (SEQ ID NO:14) |
| 02 L | TTAGACAAGTATCTTTCGGATGTG | (SEQ ID NO:15) |
| 03 L | AACGCTGCGGACATCTACATTTTTG | (SEQ ID NO:16) |
| 04 L | GTGGGTTAATTAAGAATTCAGTACATTAAA | (SEQ ID NO:17) |
| 05 L | AAGAAAATGCCGATACTTCATTGGC | (SEQ ID NO:18) |
| 06 L | AAGAAAATGCCGATACTTCATTGGC | (SEQ ID NO:19) |
| 00 R | TAGATCCGAAACTATCAGTG | (SEQ ID NO:20) |
| 02 R | ACGTGACTCCCTTTAATTCTCCGCTC | (SEQ ID NO:21) |
| 03 R | CCTAACTTTTGGTGTGATGATGCTG | (SEQ ID NO:22) |
| 04 R | TTCCCTAAATAATTCTCCGCTCATGATC | (SEQ ID NO:23) |
| 05 R | TTCCCTTAATTCTCCGCTCATGATC | (SEQ ID NO:24) |
| 06 R | TTCCCTTAATTCTCCGCTCATGATC | (SEQ ID NO:25) |
| EF U | GTTTCACATCAACATTGTGGTCATTGG | (SEQ ID NO:26) |
| EF L | GAGTACTTGGGGGTAGTGGCATCC | (SEQ ID NO:27) |

* The sequences are shown in the direction of 5′ -> 3′ according to conventional notation.

(3) Isolation of GGT Disrupted Strains

The screening for GGT in the gene disrupted Arabidopsis thaliana Library was performed using the system provided by Kazusa DNA Research Institutes. The screening was conducted by the procedure described in 2-4-c in Plant Cell Engineering Series 14 “Shokubutsu no Genome Kenkyu Protocol (Protocol of Study of Plant Genome)” (Shujunsha).

In the primary screening, AAT1U/AAT1L was used as the primer for the gene, and 00L/02L/03L/04L/05L/06L/00R/02R/03R/04R/05R/06R were used as the tag primers for the respective corresponding pools. The relationship among the tag primers and the respective pools are shown in Table 3.

TABLE 3
Relationship between tag primers and pools

| | | |
|---|---|---|
| DNA pool | Number of pools | Tag primer |

| | | | |
|---|---|---|---|
| P0009˜P0020 | 12 | 00R | 00L |
| P0023˜P0040 | 18 | | |
| P0202˜P0204 | 3 | 02R | 02L |
| P0301˜P0302 | 2 | 03R | 03L |
| P0401˜P0403 | 3 | 04R | 04L |
| P0501˜P0508 | 8 | 05R | 05L |
| P0601˜P0608 | 8 | 06R | 06L |
| Total | 54 | | |

The polymerase EX-taq (TAKARA) was used. 20 μl of the reaction solution contained about 38.4 ng (about 100 pg×384) of template DNA, 10 pmol of tag primer, 10 pmol of primer for the gene, 2 μl of 10×buffer, 5 nmol of dNTPs and 0.5 U of Ex-taq. PCR was conducted by 35 cycles of 94° C. for 45 seconds, 52° C. for 45 seconds and 72° C. for 3 minutes. Then, 10 μl of the PCR product was resolved by electrophoresis on a 1% agarose gel. The amplified DNA fragments were observed after EtBr staining. The gel was denatured by immersion in a denaturing solution (1.5 M NaCl, 0.5 M NaOH) for 20 minutes. The gel was then immersed in a neutralizing solution [0.5 M Tris-HCl (pH 8.0), 1.5 M NaCl] for 20 minutes. After blotting onto membrane-Hybond N+(Amersham Pharmacia Biotech) with 20×SSC (3M NaCl, 0.3 M sodium citrate), DNA was fixed on the membrane by UV cross-linking. The hybridization and detection were conducted with AlkPhos-Direct DNA detection kit (Amersham Pharmacia Biotech) according to the protocol attached thereto. The hybridization temperature was 65° C. PCR was conducted using primer AAT1U/AAT1L and genome DNA as a template. The amplified fragments were purified with GFX PCR DNA and Gel Band purification kit (Amersham Pharmacia Biotech).

In the primary screening, a mixture of genome DNA extracted from 384 independent tag-inserted strains was taken as one pool. 54 pools (384×54=20736 lines) were subjected to PCR. The amplification products were subjected to Southern analysis to confirm amplification of the intended product. Pool P0035 which had positive results in the primary screening was subjected to the secondary screening. The primer combination for PCR for the secondary screening was AAT1U/00L and AAT1L/00L, which gave positive results in the primary screening. By the secondary screening, it was revealed that GGT1 tag was inserted in one line, line 8046.

(4) Determination of the Location of the Tag Insertion

DNA extracted from the determined tag-inserted line was used as a template. PCR was conducted using two primer sets:AAT1U/00L, AAT1L/OOL. The amplified fragments were cloned to obtain pGEM T-easy vector (Promega). DNA sequencer, ABI PRISMTM 377 DNA sequencer (PERKIN ELMER) was used for sequencing.

It was found that the tag was inserted in the sixth exon, and 16 bp were deleted. Also,176-GGTLV-180 was replaced with 176-AIQL (end)-180 by insertion of the tag.

### Reference Example 2

### Preparation of the GGT-knockout Homozygotes

(1) Selection of Homozygotes

T2 seeds with confirmed tag insertion were placed on MS medium containing 10 mg/l of hygromycin. Three weeks later, they were transplanted on rock wools, and DNA was extracted from about 5 mm×5 mm samples of rosette leaves. The extraction was conducted according to the Li method (Plant J. 8: 457 to 463). To identify the homozygotes, PCR was conducted with the primers AAT1U/AAT1L2, which flank the tag. PCR was conducted for 30 cycles of 94° C. for 30 seconds to denature, 57° C. for 30 seconds to anneal and 72° C. for 60 seconds to elonge. For the control, a wild-type genome DNA was used as the template. An aliquot of the PCR product was resolved on 1% agarose gel by electrophoresis. In a total of 35 lines, eleven (11) lines were found to be homozygotes.

(1) Detection of GGT Expression

The obtained homozygous lines were subjected to RT-PCR by using the progenies thereof to confirm that the gene disruption had occurred. The seeds of the homozygotes were seeded on MS medium containing 10 mg/l of hygromycin, and it was confirmed that each homozygote exhibited resistance. Total RNA was extracted from seedlings with ISOGEN (Nippon gene) two weeks after seeding. After treatment with DNase followed by reverse transcription with oligo-dT primer using superscript II (GIBCO), PCR was conducted with the primers AAT1 RTU/AAT1RTL flanking the tag, and using the synthesized single-strand cDNA as a template. 28 cycles of PCR were conducted, wherein denaturation was conducted at 94° C. for 30 seconds, annealing at 57° C. for 30 seconds, and elongation at 72° C. for 60 seconds. For the control, EF1-α (EFU/EFL) was used. An aliquot of the PCR product was resolved on 1% agarose gel by electrophoresis. No full-length mRNA for GGT1 was found in the tag-inserted lines.

According to these results, the tag-inserted strain was named “ggt1-1” and used for the following analysis. The growth of the ggt1-1 strain was significantly inhibited under ordinary light strength conditions, but no significant difference was found as to the growth under weak light conditions -about 30 μmol m⁻² s⁻¹ as compared with the non-transformed plant. Additionally, it was found that the GGT activity was remarkably reduced in ggt1-1 when measured by the method described hereinafter. Therefore, ggt1-1 was used as the experimental material for increasing GGT activity.

### Example 1

### Generation of Transgenic Plants Having Increased GGT Activity (1) Introduction of the Genetic Construct for GGT Gene Expression

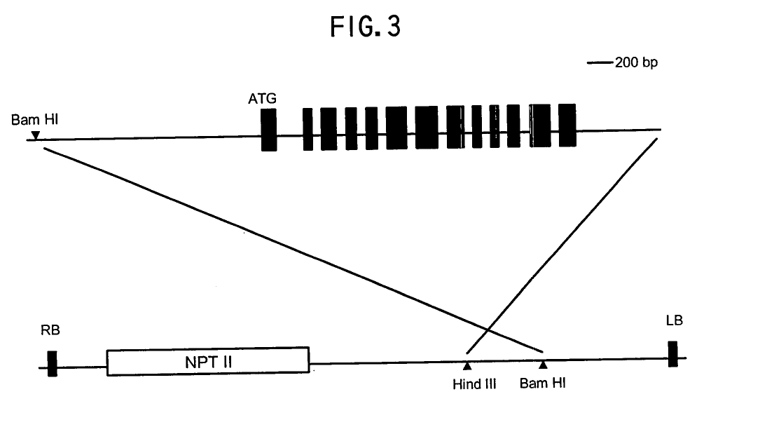

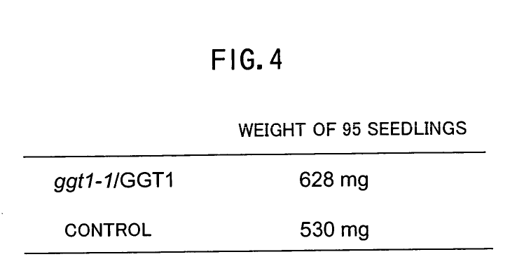

The 5089 bp genome region of GGT1 was amplified by PCR. The upstream primer was 5′-CAATAACAATGCAAAGTTAAGATTCGGATC -3′ (SEQ ID NO:28), and the downstream primer was 5′-GCTTCTTCTCAACCATCGTCACC-3′ (SEQ ID NO: 29). The nucleotide sequence encoding GGT1 and the amino acid sequence of GGT are show in SEQ ID NOs:1 and 2, and the construct of the introduced gene is shown in FIG. 3. The amplified fragment was inserted into the HindIII and BamHI site of binary vector pBI101, which has its Gus/Nos-ter deleted, and the cloned fragment was introduced into the GGT 1 gene-knockout Arabidopsis thaliana strain (ggt1-1). The resulting transformants were plated on PNS medium and were grown for 2 weeks under light conditions of 70 μmol m⁻² s⁻¹. After that, the weight of the above-ground part of the seedlings was determined. The results showed that the growth inhibition caused by the gene disruption was completely complemented, and furthermore the growth was enhanced as compared with the wild-type (FIG. 4).

(1) Confirmation of GGT Gene Expression

The expression of the introduced gene was confirmed by quantitative PCR. The seeds were plated on a ½ MS medium containing 50 mg/ml kanamycin and the lines exhibiting individual resistance were selected as a source of RNA. Total RNA was extracted from the above-ground parts of the seedlings that were grown on PNS medium for 2 weeks under light conditions of 30 μmol m⁻² s⁻¹ by using RNeasy Plant Mini Kit (QIAGEN). After DNase treatment, reverse transcription was conducted starting with an oligo dT primer using superscriptII (GIBCO) to synthesize a single strand cDNA which was in turn used as a template for PCR with the quantitative PCR primer 5′-TTCTTCTTCTGAACGACTATTGTG-3′: SEQ ID NO:30 and 5′-GAATAGGGCAAAGAGAAAGAGTG-3′: SEQ ID NO:31.

The primers 5′-GGTAACATTGTGCTCAGTGGTGG-3′: SEQ ID NO:32 and 5′-GGTGCAACGACCTTAATCTTCAT-3′: SEQ ID NO:33 were used for the quantitative PCR of ACTIN2. The quantitative PCR was conducted by ABI PRISM 7700 under the following conditions: 1 cycle of 50° C. for 2 minutes and 95° C. for 10 minutes, followed by 40 cycles of 95° C. for 15 seconds and 60° C. for 60 seconds.

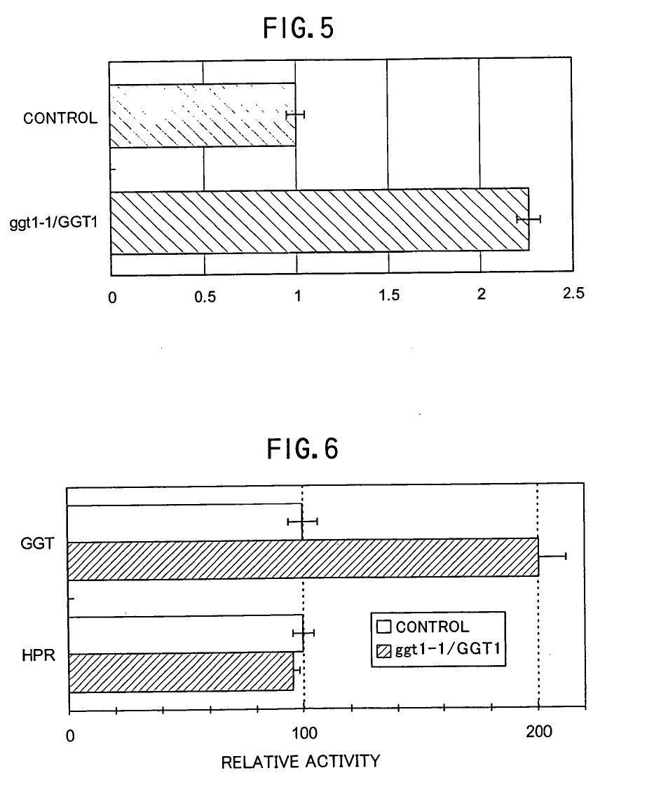

RNA was extracted and tested in triplicate for each independent experiment, and the expression of GGT1 was normalized to the expression level of ACTIN2. The quantification of GGT1 expression is shown in FIG. 5. The expression level was increased about 2-fold in the transgenic line.

### Example 2

### Evaluation of the Features of Transgenic Plants Which Have Enhanced GGT Activity

(1) Determination of GGT Enzyme Activity

To determine enzymatic activity, proteins were extracted from seedlings grown under light conditions of 70 μmol m⁻² s⁻¹ for 2 weeks after plating on PNS medium. The plant (fresh weight: about 200 mg) was frozen in liquid nitrogen and then the tissues thereof were crushed by using a mortar and pestle. 1 ml of the extraction buffer [100 mM Tris-HCl (pH 7.3), 10 mM DTT] was added thereto, and the resulting mixture was centrifuged at 15,000 rpm for 10 minutes to remove insoluble matters. This process was repeated 3 more times. The demineralization was conducted with an ultrafiltration filter UFV5BGCOO (Millipore). 0.5 ml of the extract was concentrated 10 times by centrifuging it at 10,000 rpm for about 45 minutes. After diluting the extract 10-fold, the same process was repeated 3 times. The protein concentration was determined with a protein assay kit (Bio-Rad). To obtain the crude extract, an extraction buffer containing 10% glycerol was added thereto to obtain a final concentration of 1 mg/ml of.

The activity of GGT (Glu+glyoxylate ->Gly+αKG) was determined as the change in OD at 340 nm by coupling the reaction with the oxidation reaction of NADH by NAD⁺-GDH (EC 1.4.1.3). The reaction was conducted by using 50 μg of the crude extract in 0.6 ml of the reaction solution [100 mM Tris-HCl (pH 7.3), 100 mM Glu, 0.11 mM pyridoxal 5-phosphate, 0.18 mM NADH, 15 mM glyoxylate, 500 U/I GDH (G2501)]. The activity of HPR was used for the control. The activity of HPR was determined by the change in OD at 340nm due to the oxidation of NADH. The reaction was conducted using 50μg crude extract in 0.6ml of reaction solution [100 mM Tris-HCl (pH7.3), 5 mM hydroxy pyruvate, and 0.18 mM NADH]. The activities of GGT and HPR are shown in FIG. 6. The GGT activity was found to be about 2-fold higher in the GGT transgenic lines than the corresponding non-transformed plants.

(2) Analysis of Amino Acids

To determine free amino acid content, amino acids were extracted from the seedlings grown under light conditions of 70 μmol m⁻² s⁻¹ for 2 weeks after plating on PNS medium, and also from the rosette leaf of the plants grown for 6 weeks on rock wool using PNS as a nutrient. The plant (fresh weight: about 100 mg) was frozen in liquid nitrogen and then stored at −80° C. 500 μl of 80% ethanol was added to the frozen sample, the tissue was crushed with a cell crusher MM 300 (QIAGEN), and then treated at 80° C. for 10 minutes to extract amino acids. After centrifugation at 15,000 rpm for 10 minutes, the supernatant was removed, and 500 μl of 80% ethanol was added at 80° C. to the obtained precipitate, and the mixture was thoroughly stirred and then treated at 80° C. for an additional 10 minutes. After centrifugation at 15,000 rpm for 10 minutes, the the amino acids extract, in the form of the supernatant, was taken. 1 ml of the amino acid extract was rotated under reduced pressure to completely remove ethanol and water. The sample was dissolved in 500 ml of water and the equivalent volume of diethyl ether. The lower layer after centrifugation was rotated under reduced pressure. 0.02 N HCl was added to the remaining sample to a final concentration of 10 μl/mg FW, and vortexed followed by centrifugation to recover the supernatant. The impurities were removed by passing the supernatant through a 0.22 μm filter to obtain the sample for analysis.

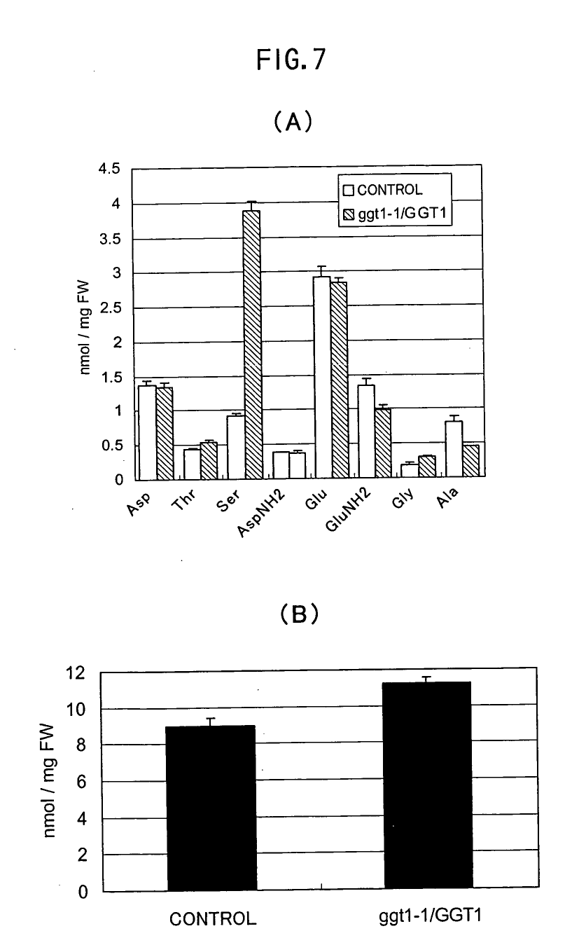

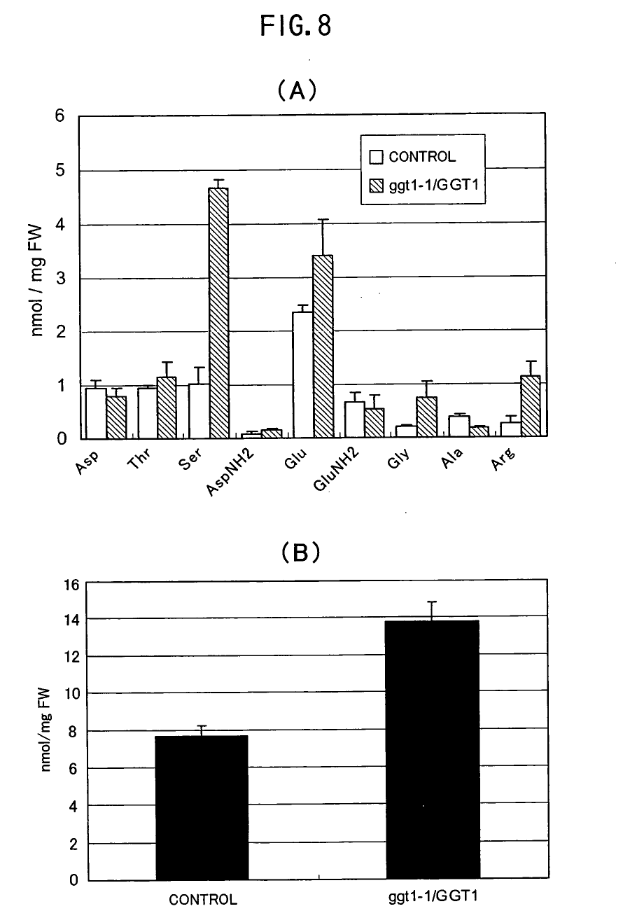

The amino acid analysis was conducted with an amino acid analyzer LS-8800 (HITACHI). The total amino acid content and individual amino acid content (nmol/mg FW) are shown in FIGS. 7 and 8. The results show that serine content was remarkably increased in the GGT1 overexpressing lines, and that the total amino acid content and the arginine content were increased in the plants grown on rock wool.

(1) Analysis of Nitrogen Content

The nitrogen content was determined in the above-ground parts of the seedlings grown under light conditions of 70 μmol m⁻² s⁻¹ for 2 weeks after seeding on PNS medium. The determination was performed using Sumigraph NC-1000 manufactured by Sumitomo Chemical Analysis Center. The results showed the nitrogen content per dry weight was increased in the GGT overexpressing strains, as indicated in Table 4.

TABLE 4
% Ratio of total nitrogen per dry weight

| | |
|---|---|
| ggt1-1/GGT1 strain | 7.21 |
| Control plant (wild-type non-transformed plant) | 7.10 |

### Example 3

### Generation of Transgenic Plants Which Have Even Greater Increased GGT Activity

To generate a plant having even greater increased GGT activity, a genetic construct for expressing GGT gene was introduced into the wild-type plant and the properties of the plant were evaluated. The GGT1-transgenic strain obtained by introducing a GGT expression construct into the GGT1 gene disrupted strain (ggt1-1) is hereinafter referred to as “ggt1-1 /GGT1” strain, and the GGT1-transgenic strain obtained by introducing a GGT expression construct into the wild-type plant is hereinafter referred to as “WT/GGT1” strain.

Introduction of the Genetic Construct for GGT Gene Expression

GGT1 gene was introduced to the wild type (Col-0) by using proceduressimilar to those described in Example 1 (1).

(2) Confirmnation of Expression of the GGT Gene

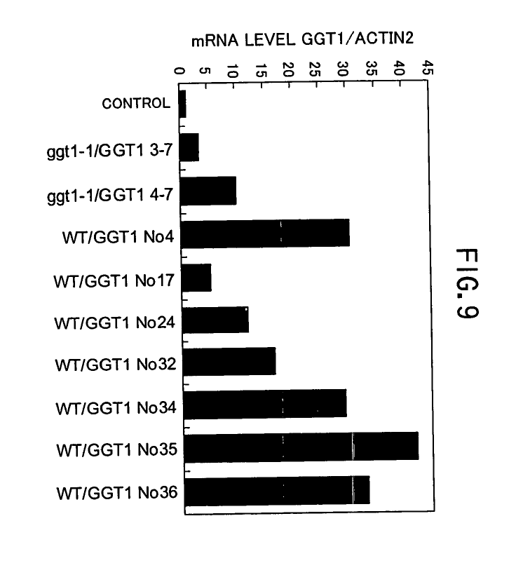

The expression of the transgene was confirmed by the method described in Example 1 (2). The wild-type strain which had been grown for 2 weeks on PNS medium, 2 lines from ggt1-1/GGT1, and seven lines from WT/GGT1 were used as the source of RNA. The quantification of GGT1 expression is shown in FIG. 9. The expression was increased 5- to 30-fold in the transgenic lines.

### Example 4

### Evaluation of the Transgenic Plants Having Much Greater Enhanced GGT Activity

Determination of GGT Enzyme Activity

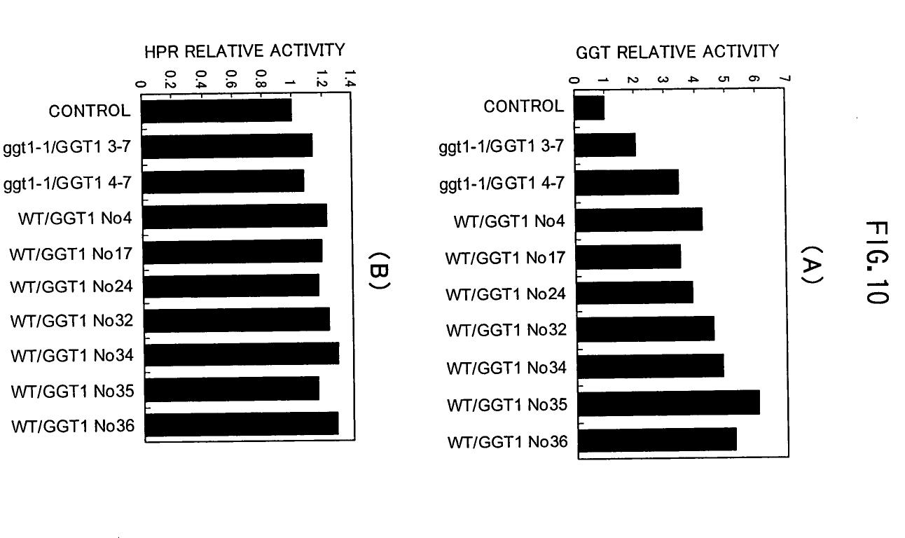

The determination of the enzyme activity was conducted by the methods described in Example 2 (1). GGT activity and the control HPR activity were shown in FIG. 10. The GGT activity was increased about 2- to 6-fold in the GGT transgenic lines as compared with the wild-type.

(2) Amino Acid Analysis

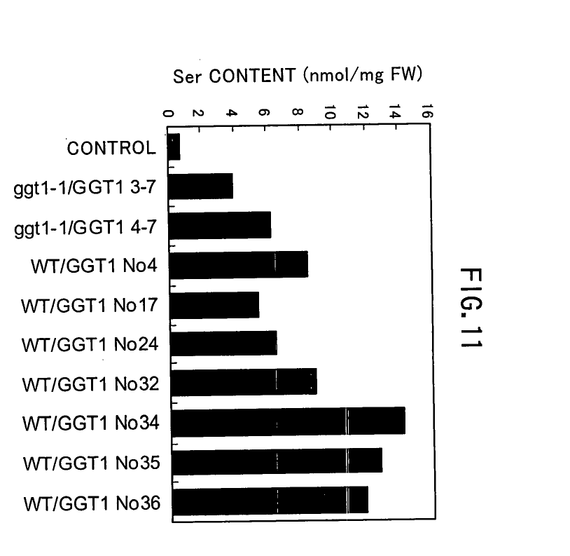

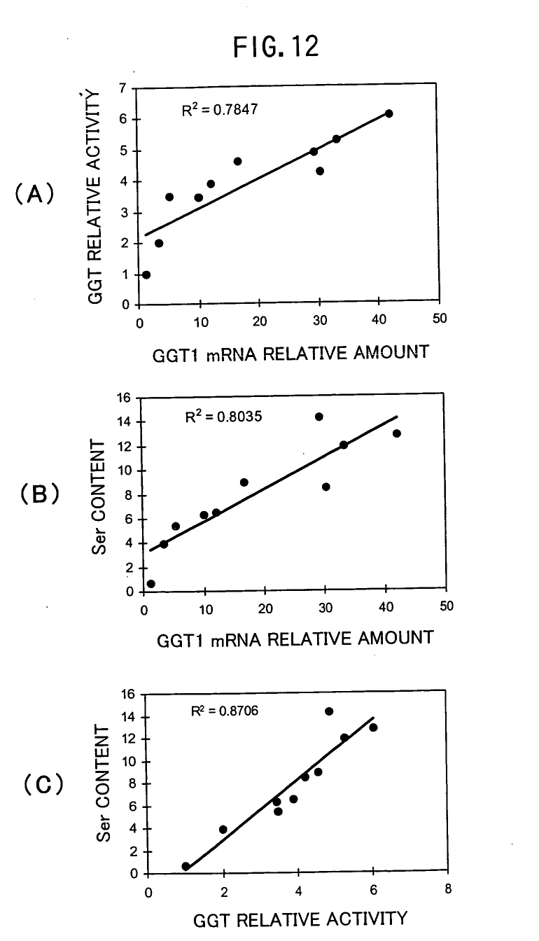

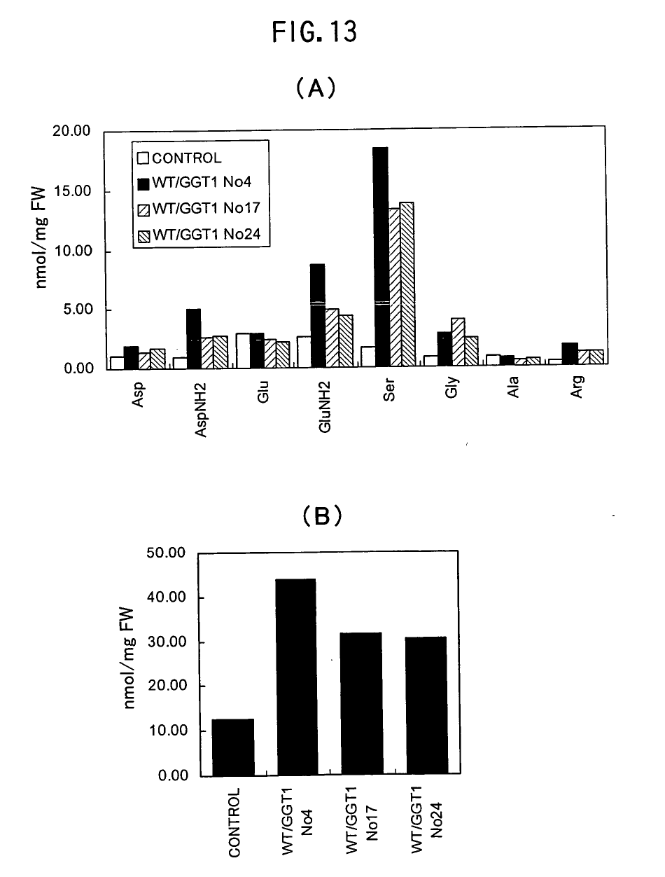

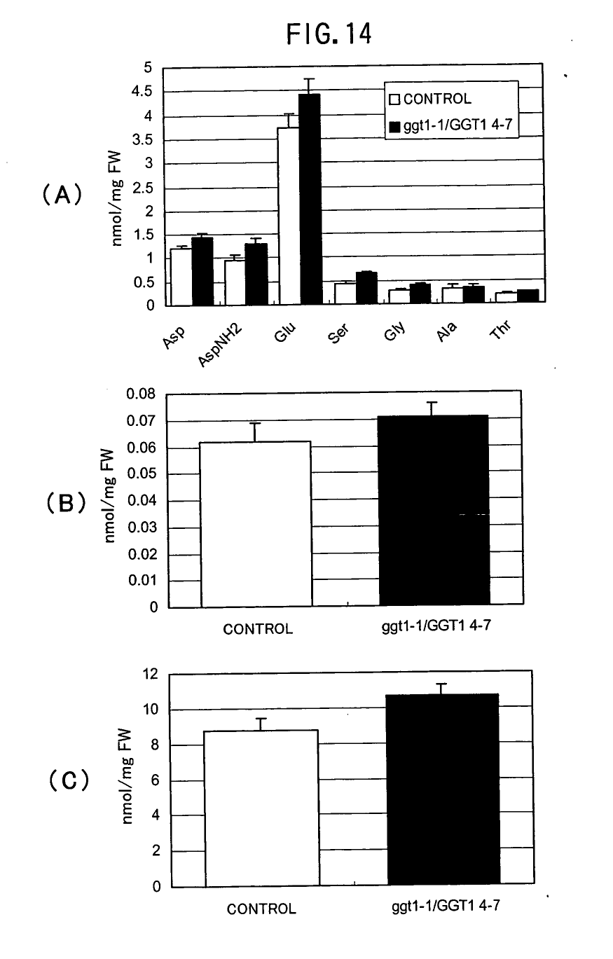

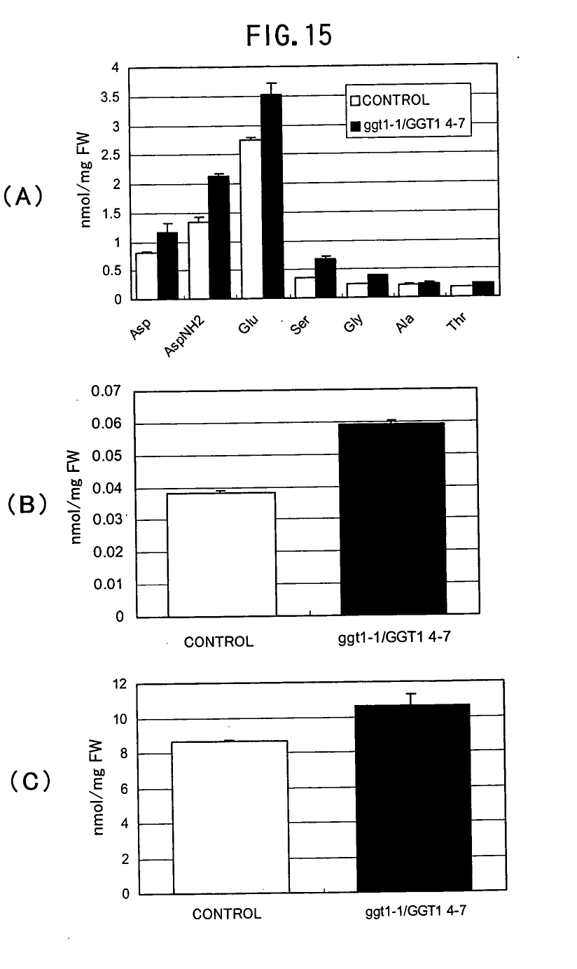

The content of free amino acids was determined by the method described in Example 2 (2). The serine content (nmol/mg FW) of the strains of which GGT expression level and the enzyme activities were determined in Example 3(2) and Example 4 (1) are shown in FIG. 11 for PNS medium cultivation. The determined results obtained from 40 lines in total were shown in Table 5. The relationship between expression, enzyme activity, and serine content are shown in FIG. 12. Individual amino acid content and the total amino acids of the plants grown on ½ MS medium are shown in FIG. 13. The amino acid content of seeds is shown in FIGS. 14 and 15. The results of the analysis showed that the serine content increased up to 20-fold in the GGT1 overexpressing lines. The comparison of expression, enzyme activity, and serine content revealed that they had a significant relationship each other.

TABLE 5
Ser Content

| Line | Ser Content (nmol/mgFW) |
|---|---|
| Control | 0.69 |
| WT/GGT1 No. 1 | 7.43 |
| WT/GGT1 No. 2 | 2.14 |
| WT/GGT1 No. 3 | 7.33 |
| WT/GGT1 No. 4 | 8.42 |
| WT/GGT1 No. 5 | 7.68 |
| WT/GGT1 No. 6 | 10.07 |
| WT/GGT1 No. 7 | 5.84 |
| WT/GGT1 No. 8 | 4.54 |
| WT/GGT1 No. 9 | 10.13 |
| WT/GGT1 No. 10 | 8.51 |
| WT/GGT1 No. 11 | 3.03 |
| WT/GGT1 No. 12 | 8.01 |
| WT/GGT1 No. 13 | 4.84 |
| WT/GGT1 No. 14 | 3.07 |
| WT/GGT1 No. 15 | 6.97 |
| WT/GGT1 No. 16 | 6.92 |
| WT/GGT1 No. 17 | 5.44 |
| WT/GGT1 No. 18 | 7.41 |
| WT/GGT1 No. 19 | 9.06 |
| WT/GGT1 No. 20 | 4.01 |
| WT/GGT1 No. 21 | 8.20 |
| WT/GGT1 No. 22 | 3.20 |
| WT/GGT1 No. 23 | 5.92 |
| WT/GGT1 No. 24 | 6.53 |
| WT/GGT1 No. 25 | 5.42 |
| WT/GGT1 No. 26 | 8.66 |
| WT/GGT1 No. 27 | 1.48 |
| WT/GGT1 No. 28 | 7.37 |
| WT/GGT1 No. 29 | 7.32 |
| WT/GGT1 No. 30 | 11.66 |
| WT/GGT1 No. 31 | 8.06 |
| WT/GGT1 No. 32 | 8.91 |
| WT/GGT1 No. 33 | 8.19 |
| WT/GGT1 No. 34 | 14.25 |
| WT/GGT1 No. 35 | 12.80 |
| WT/GGT1 No. 36 | 11.89 |
| WT/GGT1 No. 37 | 11.28 |
| WT/GGT1 No. 38 | 7.01 |
| WT/GGT1 No. 39 | 5.01 |
| WT/GGT1 No. 40 | 3.43 |

When the plants were grown on ½ MS medium, asparagine increased about 5-fold, glutamine increased about 3-fold, arginine increased about 5-fold, and the total amino acids increased about 4-fold in Strain No.4 as compared with the wild-type. Furthermore, in the GGT1 overexpression lines, Ser content was remarkably increased when the lines were grown on PNS medium and, other than Ser, asparagine, glutamine and arginine, were increased about 3- to 5-fold when the lines were grown on ½ MS medium containing ammonia-nitrogen.

The amino acids were determined in the seeds from plants grown under light conditions of about 200 μmol m⁻² s⁻¹ continuous light on the modified PNS (5 mM KNO₃ was replaced with 2.5 mM NH₄NO₃). Asparagine, aspartate, glutamate, serine, glycine, and arginine accumulated and increased in ggt1-1/GGT1 No. 4-7 line as compared with the wild-type. The total amino acids were also increased.

### Example 5

### Generation of Tomato GGT and Potato GGT Transformants

Generation of Tomato Transformants

Seeds of tomato (cultivar, Mini-tomato Fukukaenn-Shubyou) are surface-sterilized by 70% ethanol (30 seconds) and 2% sodium hypochloride (15 minutes), placed on plant hormone-free MS-agar plates and grown at 25° C. for 1 week under 16-hour daylight. The cotyledons are picked up from the resulting sterile seedlings and placed on MS agar plates containing 2 mg/ml zeatin and 0.1 mg/ml indoleacetate (regeneration medium, 9 cm dish), and further cultivated for 2 days under said condition. The Agrobacterium EHA101 harboring the constructed gene are grown in YEP medium (Table 6) overnight and used for infection. The cotyledons that have been cultured for 2 days are collected in a dish and the Agrobacterium suspension was added for infection. A sterile filter is used for removing the Agrobacterium suspension from the cotyledons and the infected cotyledons were placed on the sterile filter which is placed on the aforementioned medium plate to avoid the rapid growth of the agrobacteria. The cotyledons are co-cultured for 24 hours.

After the period, the cotyledons are transferred onto a MS regeneration medium (selection medium) containing 50 mg/ml kanamycin and 500 mg/ml Claforan to select the transformants. The regenerated shoots are transferred to a fresh selection medium for re-selection. The vigorously growing green shoots are cut at the stems and placed on the MS medium (rooting medium, in tubes) which is free of plant hormones. The rooted regenerated plants are continuously acclimated to soils.

TABLE 6
YEP medium composition

| YEP medium ingredients | (1 liter) |
|---|---|
| Bacto Trypton | 10 g |
| Yeast Extract | 10 g |
| Glucose | 1 g |

(2) Generation of Potato Transformants

The sterile potato plants were obtained by stem apex culture and the materials were increased by subculturing the stem apexes. The stem apexes were induced for rooting by placing them into MS liquid medium (10 ml) supplemented with 2% sucrose. After rooting, 10 ml of MS liquid medium containing 16% sucrose was added and the stem apexes were cultured in a dark place to induce microtubers. The microtubers of 6-8 weeks culture were sectioned into discs, pealed, and were infected with agrobacteria into which the genetic construct described in Example 1 (1) had been introduced and which had been grown overnight at 28° C. The discs are placed on a sterile filter which is laid on a MS agar plate (MS medium, 2.0 mg/ml zeatin, 0,1 mg/l indoleacetate, 0.3% gelite) and are co-cultured for 2 days at 25° C. under 16-hours daylight. Then the discs are transferred to a selection medium [Ms medium, 2.0mg/ml zeatin, 0,1 mg/l indole acetate, 0.3% gelite, 50 mg/l kanamycin, 500 mg/l Claforan] and cultured under the same conditions. They are transferred into a fresh selection medium at one week intervals the regenerated shoots are transferred to a selection medium which do not contain plant hormones to induce rooting. They are infected with the agrobacteria into which the genetic construct described in Example 1 (1) has been introduced and are selected on a medium containing 50 mg/ml of kanamycin.

### Example 6

### Generation of Rice GGT Transformants

Generation of Arabidopsis thaliana GGT1 Gene Introduced Rice

The cDNA of Arabidopsis thaliana GGT1 gene region was amplified by PCR method. The primer 5′-GCGGATCCATGGCTCTCAAGGCATTAGACT-3′: SEQ ID NO:38 was used for the upstream primer and 5′-GCCGAGCTCTCACATTTTCGAATAA-3′: SEQ ID NO:39 was used for the downstream primer. The amplified fragment was linked downstream of the CAB promoter (Plant Cell Physiol 42, 138-, 2001) using the underlined restriction enzyme site (BamHII, Sacd) to replace the 35S promoter+GUS region in the binary vector pIG121HM. This was introduced into a rice plant (race=Kiatake) through Agrobacterium. The transformation was conducted according to the Method of Toriyama et al. (Experimental Protocols for Model Plants, 93-, 1996, Shujunsha).

The individual plants that exhibited resistance on a selection medium containing hygromycin were transferred into soil and the leaves were sampled for RNA extraction and amino acid analysis.

(2) Confirmation of GGT1 Gene Expression

The expression of the transgene was confirmed by RT-PCR for 20 strains that had been selected for the drug resistance. Total RNA was extracted using RNeasy Plant Mini Kit (QIAGEN). After DNase treatment, reverse transcription was conducted with oligo dT primer using superscript II (GIBCO) and the synthesized single strand cDNA was used as a template for PCR using PCR primers (5′-TGAAAGCAAGGGGATTCTTG-3′: SEQ ID NO:40 and 5′-GACGTTTTTGCAGCTGTTGA-3′: SEQ ID NO: 41). The reaction was performed under the following conditions: 40 cycles of 95° C. for 15 seconds, 60° C. for 60 seconds. The amplification of GGT1 DNA fragment was confirmed in the tested 20 lines of transformant, which was indicative of expression of the transgene.

(3) Amino Acid Content of the GGT1 Transgenic Rice

The determination of free amino acid content was performed according to the method described in Example 2 (2). It was shown that Serine was significantly increased in the transformants as compared with the non-transformants (FIG. 17).

Sequence listing:
SEQ ID NOs:9-33, 38-41: PCR primer

According to the present invention, a novel method for utilizing glutamate glyoxylate amino transferase (GGT) for improving the properties of plants.

According to the present invention, a plant having increased GGT activity is provided. Particularly, according to the present invention a plant having increased GGT activity preferably more than about 2-fold, more preferably more than about 3-fold, and most preferably more than about 5-fold.

Additionally, according to the present invention, a method of increasing the amino acids content in a plant and/or a seed, particularly a method of increasing at least one of Ser, Arg, Gln and Asn, a plant and/or a seed having increased amino acids contents, particularly a plant and/or a seed where at least one of Ser, Arg, Gln and Asn content is increased, the use of such plants and/or seeds for the production of feeds and a feed containing a plant and/or a seed having increased glutamate content.

A plant extract containing at least one of the amino acid Ser, Arg, Gln and Asn in a large amount can be easily obtained according to the present invention.

Furthermore, it has been suggested that there is a strong correlation between the lysine content and the content of glutamine, glutamate, asparagine and aspartate (Plant Cell 15, 845-853, 2003). Therefore, the plant of the present invention or the method of producing such plants may also provide a plant having increased lysine content.

While the invention has been described in detail with reference to preferred embodiments thereof, it will be apparent to one skilled in the art that various changes can be made, and equivalents employed, without departing from the scope of the invention. Each of the aforementioned documents, including the foreign priority documents, JP2002-232562 filed on Aug. 9, 2002 and JP2003-194431 filed on Jul. 9, 2003, is incorporated by reference herein in its entirety.

1. A plant comprising increased glutamate glyoxylate aminotransferase (GGT) activity as compared with a wild-type plant of the same species which is cultivated under the same conditions.

2. The plant according to claim 1, comprising increased amino acid content as compared with the wild-type plant of the same species which is cultivated under the same conditions.

3. The plant according to claim 1, wherein the content of at least one of the amino acids selected from the group consisting of serine, arginine, glutamine, and asparagine is increased as compared with the wild-type plant of the same species which is cultivated under the same conditions.

4. The plant according to claim 1, wherein said increase in GGT activity is a result of an increase in the copy number of the GGT gene.

5. The plant according to claim 1, wherein said increase in GGT activity is a result of an increase in an mRNA level, wherein said mRNA corresponds to the GGT gene.

6. The plant according to claim 1, wherein said GGT activity is in a peroxisome.

7. The plant according to claim 1, wherein said GGT activity is the activity of a protein having at least 60% homology to the amino acid sequence selected from the group consisting of SEQ ID NO:2 and SEQ ID NO:4.

8. The plant according to claim 6, wherein said GGT activity is the activity of a protein having the amino acid sequence selected from the group consisting of SEQ ID NO:2 and SEQ ID NO:4.

9. A transgenic plant comprising increased GGT activity as compared with a corresponding non-transformed plant which is cultivated under the same conditions, wherein a genetic construct capable of increasing the expression of a GGT gene is introduced into said transgenic plant.

10. The transgenic plant according to claim 9, comprising increased amino acid content as compared with a corresponding non-transformed plant of the same species which is cultivated under the same conditions.

11. The transgenic plant according to claim 9, wherein the content of at least one of the amino acids selected from the group consisting of serine, arginine, glutamine, and asparagine is increased as compared with a corresponding non-transformed plant which is cultivated under the same conditions.

12. The transgenic plant according to claim 9, wherein the genetic construct is capable of increasing the expression of a GGT gene.

13. The transgenic plant according to claim 9, wherein said genetic construct being capable of increasing the expression of a GGT gene contains a genetic construct being capable of increasing a transcription level of the GGT gene.

14. The transgenic plant according to claim 12, wherein the GGT gene has a nucleotide sequence which is capable of hybridizing to the polynucleotide of SEQ ID NO: 1 or SEQ ID NO:3 under stringent conditions.

15. The transgenic plant according to claim 9, wherein the GGT activity is in a peroxisome.

16. The transgenic plant according to claim 12, wherein the GGT gene comprises a nucleotide sequence selected from the group consisting of SEQ ID NO: 1 and SEQ ID NO:3.

17. A method of increasing amino acid content in a plant or in a plant seed comprising transforming into said plant or seed a genetic construct capable of increasing the expression of a GGT gene, wherein said genetic construct can increase the GGT activity in said plant or seed as compared with a corresponding non-transformed plant or seed which is cultivated under the same conditions.

18. The method according to claim 17, wherein the content of at least one amino acid selected from the group consisting of serine, arginine, glutamine and asparagine in said plant or seed is increased.

19. The method according to claim 17, wherein the genetic construct is capable of expressing the GGT gene.

20. The method according to claim 17, wherein said genetic construct is capable of increasing a transcription level of the GGT gene.

21. The method according to claim 19, wherein the GGT gene has the nucleotide sequence which is capable of hybridizing to the polynucleotide selected from the group consisting of SEQ ID NO:1 and SEQ ID NO:3 under a stringent conditions.

22. The method according to claim 17, wherein said GGT activity is in a peroxisome.

23. The method according to claim 19, wherein said GGT gene has the nucleotide sequence selected from the group consisting of SEQ ID NO:1 and SEQ ID NO:3.

24. A seed of said transgenic plant according to claim 9 comprising.

25. A method of producing a plant comprising a step selected from the group consisting of:
i) germinating a seed of the plant according to claim 1,
ii)

26. A method of producing a plant comprising germinating the seed of claim 24.

27. A method of producing a plant comprising regenerating a plant from a cell of the plant of claim 1.

28. A method of producing a plant comprising performing vegetative propagation of the plant according to claim 1.

29. A feed produced from a plant according to claim 1.

30. A feed produced from a seed according to claim 24.

31. A method of producing at least one amino acid selected from the group consisting of serine, arginine, glutamine, and asparagine comprising recovering said amino acid from the plant according to claim 1 or from the seeds according to claims 18.

32. A method of producing a plant extract containing at least one amino acid selected from the group consisting serine, arginine, glutamine, and asparagine comprising recovering the plant extract containing said amino acid(s) from the plant according to claim 1.

33. A method of producing at least one amino acid selected from the group consisting of serine, arginine, glutamine, and asparagine comprising recovering said amino acid from the seed according to claim 24.

34. A method of producing a plant extract containing at least one amino acid selected from the group consisting serine, arginine, glutamine, and asparagine comprising recovering the plant extract containing said amino acid(s) from the seed according to claim 24.

35. The transgenic plant according to claim 9, wherein the expression of the genetic construct is under the control of a CAB promoter.

36. The transgenic plant according to claim 9, wherein the expression of the genetic construct is under the control of a native promoter of GGT.

37. The transgenic plant according to claim 17, wherein the expression of the genetic construct is under the control of a CAB promoter.

38. The transgenic plant according to claim 17, wherein the expression of the genetic construct is under the control of a native promoter of GGT.
